SEQUENCE LISTING (19 sequences)

SEQ ID NO: 1 - protein, 8 aa, synthetic construct
GYTFTDYN

SEQ ID NO: 2 - protein, 10 aa, synthetic construct
HINPNNGGTF

SEQ ID NO: 3 - protein, 8 aa, synthetic construct
GGGLRRGP

SEQ ID NO: 4 - protein, 14 aa, synthetic construct
ASESFDSYGNTFVH

SEQ ID NO: 5 - protein, 6 aa, synthetic construct
LVSNLE

SEQ ID NO: 6 - protein, 9 aa, synthetic construct
QQNNEDPYT

SEQ ID NO: 7 - protein, 119 aa, synthetic construct
EVQLQQSGPELVKPGASVKIPCKASGYTFTDYNMDWMKQSHGKSLEWIGHINPNNGGTFYNQKFKDKATFIVDKSSNTAYMELRSLTSEDTAVYFCARGGGLRRGPFAYWGQGTLVTVS

SEQ ID NO: 8 - protein, 112 aa, synthetic construct
DIVTQSPASLAVSLGQRATISCRASESFDSYGNTFVHWYQQKPGQPPKLLIYLVSNLESGVPAGFRGRGSRTDFTLTIDPVEADDAATYYCQQNNEDPYTFGGGTKLEIKRA

SEQ ID NO: 9 - protein, 267 aa, Homo sapiens
MRLTVLCAVCLLPGSLALPLPQEAGGMSELQWEQAQDYLKRFYLYDSETKNANSLEAKLKEMQKFFGLPITGMLNSRVIEIMQKPRCGVPDVAEYSLFPNSPKWTSKVVTYRIVSYTRDLPHITVDRLVSKALNMWGKEIPLHFRKVVWGTADIMIGFARGAHGDSYPFDGPGNTLAHAFAPGTGLGGDAHFDEDERWTDGSSLGINFLYAATHELGHSLGMGHSSDPNAVMYPTYGNGDPQNFKLSQDDIKGIQKLYGKRSNSRKK

SEQ ID NO: 10 - DNA, 22 bp, synthetic construct
  source 1..22: mol_type = other DNA
atatcgcggtgtaaaactcgtc

SEQ ID NO: 11 - DNA, 21 bp, synthetic construct
  source 1..21: mol_type = other DNA
taggcttaggcgtttctggaa

SEQ ID NO: 12 - DNA, 20 bp, synthetic construct
  source 1..20: mol_type = other DNA
gcagaggcatacttgtaccg

SEQ ID NO: 13 - DNA, 23 bp, synthetic construct
  source 1..23: mol_type = other DNA
tgatgttatgatggtcccacttg

SEQ ID NO: 14 - DNA, 23 bp, synthetic construct
  source 1..23: mol_type = other DNA
tgatgttatgatggtcccacttg

SEQ ID NO: 15 - DNA, 21 bp, synthetic construct
  source 1..21: mol_type = other DNA
gggagagttttccagtcatgg

SEQ ID NO: 16 - DNA, 21 bp, synthetic construct
  source 1..21: mol_type = other DNA
aggctgatttggcaaccatga

SEQ ID NO: 17 - DNA, 23 bp, synthetic construct
  source 1..23: mol_type = other DNA
cccaccttaggggtgtaattctg

SEQ ID NO: 18 - DNA, 22 bp, synthetic construct
  source 1..22: mol_type = other DNA
cacaaaacttgagagtcgtggt

SEQ ID NO: 19 - DNA, 21 bp, synthetic construct
  source 1..21: mol_type = other DNA
gctagaaccctagagtcaggc

(19) United States
(12) Patent Application Publication
(10) Pub. No.: US 2026/0286008 A1
(43) Pub. Date: Sep. 24, 2026

(54) COMPOSITIONS FOR USE IN THE TREATMENT OF ENDOMETRIOSIS

(71) Applicant: YEDA RESEARCH AND DEVELOPMENT CO. LTD., Rehovot (IL)

(72) Inventors: Irit SAGI, Rehovot (IL); Tamar GROSS, Rehovot (IL)

(21) Appl. No.: 19/477,835

(22) PCT Filed: Apr. 21, 2024
(86) PCT No.: PCT/IL2024/050401

§ 371 Date: Oct. 23, 2025

(30) Foreign Application Priority Data
Apr. 23, 2023 (IL) 302363

(51) Int. Cl. C07K 16/40; A61P 15/00; C12Q 1/6883; G01N 33/68

(52) U.S. Cl. CPC C07K 16/40; A61P 15/00; C12Q 1/6883; G01N 33/689; C07K 2317/24; C07K 2317/92; C12Q 2600/158; G01N 2333/96494; G01N 2800/364

(57) ABSTRACT

The present invention is directed to a pharmaceutical composition including a therapeutically effective amount of an agent capable of inhibiting an active site of a matrix metalloprotease (MMP-7) protein, and use of same in a method for treating endometriosis or ameliorating at least one symptom associated therewith, in a subject in need thereof.

### REFERENCE TO AN ELECTRONIC SEQUENCE LISTING

The contents of the electronic sequence listing (YEDA-P-024-PCT ST26.xml; size: 18,081 bytes; and date of creation: Apr. 1, 2024) is herein incorporated by reference in its entirety.

### CROSS-REFERENCE TO RELATED APPLICATIONS

The application claims the benefit of priority of Israel Patent Application No. 302363, titled “COMPOSITIONS FOR USE IN THE TREATMENT OF ENDOMETRIOSIS”, filed 23 Apr. 2023, the contents of which are incorporated herein by reference in their entirety.

### FIELD OF THE INVENTION

The present invention relates to, inter alia, compositions and methods of using same for treating endometriosis or a symptom associated therewith.

### BACKGROUND OF THE INVENTION

Endometriosis, a pathological condition that involves mechanisms of cells and tissue implantation into existing tissues, is defined as the presence of functional endometrial glands and stroma outside the uterine cavity, such as peritoneum, ovaries and rectovaginal septum. Retrograde menstruation is the most widely accepted theory regarding the etiology of the disease. Accordingly, endometriosis develops as a consequence of the reflux of endometrial fragments through the fallopian tubes during menstruation that subsequently undergoes implantation and growth on and into organs in the peritoneum cavity. The development of the endometriotic implants requires escape from immune clearance, attachment to peritoneal surface, invasion to the basement membrane and extracellular matrix (ECM), acquirement of blood supply and continued growth and survival. Previous studies have revealed striking differences in gene and protein expression comparing eutopic endometrium (endometrium from its correct place in the uterus) from women with and without endometriosis that may predispose the latter to develop the disease, among them are members from the matrix metalloproteinase (MMP) family.

Many studies have investigated the change in expression of various MMPs during endometriosis. These studies showed that the expression of most MMPs (MMP-1, 2, 3, 9, 11, 14, and 7) is elevated while the expression of tissue inhibitors of metalloproteinases (TIMPs) is reduced in endometriotic lesions or in the endometrium of woman with endometriosis compared to healthy women. However, those studies are only descriptive at the level of MMP expression, and the functional involvement of MMPs in the development or progression of endometriosis was not shown. Thus, whether the upregulated MMP expression and activity in the ectopic endometrium or peritoneal fluid of women with endometriosis is a cause or a consequence of the disease, or if it is functionally relevant in established lesions remains to be determined.

Therefore, there is still a great need for insight on the functional role of MMPs in endometriosis, and compositions and methods for inhibiting same, for the treatment of endometriosis.

### SUMMARY OF THE INVENTION

The present invention, in some embodiments, is based, at least in part, on the surprising findings that a monoclonal antibody that is highly selective and specific inhibitor for active MMP-7, significantly decreased the weight of endometriotic lesions in vivo. In sharp contrast, monoclonal antibodies specifically inhibiting other MMP enzymes, i.e., MMP-9 and TACE did not significantly reduce the weight of endometriotic lesions in vivo.

According to one aspect, there is provided a pharmaceutical composition comprising a therapeutically effective amount of an agent capable of inhibiting an active site of a matrix metalloprotease (MMP-7) protein, for use in the treatment of endometriosis or amelioration of at least one symptom associated therewith, in a subject in need thereof.

According to another aspect, there is provided a pharmaceutical composition comprising a therapeutically effective amount of an agent capable of inhibiting an active site of an MMP-7 protein, for use in the treatment of a subject afflicted with endometriosis or being afflicted with at least one symptom associated therewith, and determined as having at least one endometrial lesion characterized by increased MMP-7 expression level compared to a healthy control subject.

In some embodiments, the MMP-7 expression level is determined in a biopsy sample obtained or derived from the subject.

In some embodiments, the MMP-7 expression level comprises mRNA expression level, protein level, or both.

In some embodiments, inhibiting is by binding to at least one amino acid residue selected from the group consisting of: L181, A216, Y241, P246, Q247, N243, Y172, T180, P237, T240, H229, S101, N179, and any combination thereof, of the active site of the MMP-7 protein.

In some embodiments, the agent is an antibody or an antigen-binding portion thereof.

In some embodiments, the antibody or an antigen-binding portion thereof comprises three heavy chain complementarity determining regions (CDR-H) and three light chain CDRs (CDR-L), wherein: CDR-H1 comprises the amino acid sequence: GYTFTDYN (SEQ ID NO: 1), CDR-H2 comprises the amino acid sequence: HINPNNGGTF (SEQ ID NO: 2), CDR-H3 comprises the amino acid sequence: GGGLRRGP (SEQ ID NO: 3), CDR-L1 comprises the amino acid sequence: ASESFDSYGNTFVH (SEQ ID NO: 4), CDR-L2 comprises the amino acid sequence: LVSNLE (SEQ ID NO: 5), and CDR-L3 comprises the amino acid sequence: QQNNEDPYT (SEQ ID NO: 6).

In some embodiments, the antibody or antigen-binding portion thereof comprises a heavy chain amino acid comprising the sequence:

(SEQ ID NO: 7)
EVQLQQSGPELVKPGASVKIPCKASGYTFTDYNMDWMKQSHGKSLEWIGH
INPNNGGTFYNQKFKDKATFIVDKSSNTAYMELRSLTSEDTAVYFCARGG
GLRRGPFAYWGQGTLVTVS.

In some embodiments, the antibody or antigen-binding portion thereof comprises a light chain amino acid comprising the sequence:

(SEQ ID NO: 8)
DIVTQSPASLAVSLGQRATISCRASESFDSYGNTFVHWYQQKPGQPPKLL
IYLVSNLESGVPAGFRGRGSRTDFTLTIDPVEADDAATYYCQQNNEDPYT
FGGGTKLEIKRA.

In some embodiments, inhibiting is with an inhibition constant (Ki) of between 10 and 200 nM.

In some embodiments, the binding is with a dissociation constant (Kd) of between 30 and 50 nM.

In some embodiments, the pharmaceutical composition is formulated for intravenous administration.

In some embodiments, the antibody is a humanized monoclonal antibody.

In some embodiments, the subject comprises at least one endometrial lesion characterized by increased amount of at least one cell type selected from the group consisting of: an epithelial cell, a T cell, a myofibroblast, a mature blood cell, an endothelial progenitor cell, and any combination thereof, compared to a healthy control subject.

In some embodiments, the: epithelial cell, T cell, mature blood cell, endothelial progenitor cell, myofibroblast, or any combination thereof, is characterized by increased expression of a gene encoding MMP-7, compared to a control cell.

In some embodiments, the mature blood cell is characterized by expression of CD31 (CD31+).

In some embodiments, the endothelial progenitor cell is characterized by expression of CD34 (CD34+).

In some embodiments, the treated subject is characterized by reduced: number of endometriotic lesions, rate of implantation of endometriotic lesions, weight of endometriotic lesions, or any combination thereof.

In some embodiments, the treated subject comprises at least one endometrial lesion characterized by increased expression of at least one gene associated with any one of: eosinophil migration, T cell migration, cellular defense response, myeloid dendritic cell activation, chemokine binding, lymphocyte migration, antigen processing and presentation of exogenous peptide, chemokine-mediated signaling pathway, immune response-regulating cell surface receptor signaling pathway, immunological synapse, myeloid leukocyte activation, any combination thereof, compared to a non-treated control subject.

In some embodiments, the antigen processing and presentation of exogenous peptide comprises processing and presentation via MHC class II.

In some embodiments, the treated subject comprises at least one endometrial lesion characterized by decreased expression of at least one gene associated with any one of: epithelial cell differentiation, glycolysis and/or gluconeogenesis, glucose metabolic process, cellular carbohydrate biosynthetic process, cell-cell adherence junction, tight junction, glucan biosynthetic process, junctional membrane complex, and any combination thereof, compared to a non-treated control subject.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The subject matter being regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIGS. 1A-1B include a scheme and micrographs showing the establishment of an intraperitoneal (I.P.) and subcutaneous (S.C.) in-vivo endometriosis models. (1A) A chronological non-limiting scheme describing the in vivo I.P. and S.C. endometriosis exo-transplantation models. (1B) Images of implants excised at days 5, 14 and 21, in both models.

FIG. 2 includes micrographs and fluorescent micrographs showing histological characterization of the S.C. model. Representative images of hematoxylin and eosin (H&E) histochemical stain (left column) including magnification on the border area (middle column), and immunofluorescence stains of anti-green fluorescent protein (GFP; right column).

FIG. 3 includes micrographs and fluorescent micrographs showing histological characterization of the I.P. model. Representative images of H&E histochemical stain (left column) including magnification on the border area (middle column), and immunofluorescence stains of anti-GFP (right column).

FIGS. 4A-4D include fluorescent micrographs and vertical bar graphs showing angiogenic characterization of the I.P. and S.C. models. (4A) Representative images of immunofluorescence stain with anti-CD31 at different time points in the S.C. model, including magnification on representative area of CD31 expression. The yellow dash lines represent a lesioned area. (4B) Covered area quantification of CD31 inside the lesion area in the S.C. model. (4C) Representative images of immunofluorescence stain with anti-CD31 at different time points in the I.P. model, including magnification on representative area of CD31 expression. The yellow dash lines represent the lesioned area. (4D) Covered area quantification of CD31 inside the lesion area in the I.P. model.

FIGS. 5A-5C include graphs showing inflammatory characterization of the S.C. model. (5A) qRT-PCR analysis of the immune marker CD45 at different time points (n=4-6). (5B) Comparison between day 3 and day 21 based on the mass cytometry analysis depicting the changes in the relative frequencies (out of all live cells) of the indicated cell populations (n=2, 3 mice per group). (5C) Cluster t-SNE of cell lineages implied from expression of marker genes which were examine in a cell isolation and mass cytometry (cyTOF) experiments of day 3 and 21 lesions (n=2, 3 mice per group).

FIGS. 6A-6C include graphs and micrographs showing proteases characterization of the S.C. model. (6A) qRT-PCR analysis of matrix metalloprotease (MMP)-9, MMP-7, MMP-14, and TACE at different time points (n=4-6). Day 0 represent donor tissue. (6B) Western-blot (WB) with anti MMP-9, MMP-7, MMP-14, and TACE antibodies of lesion samples at different time points. Protease expression was normalized to a-tubulin expression. Quantification of the WB was done using ImageJ software (n=2-3). (6C) cyTOF analysis of lesions at day 3 and 21. Graph present the analysis of cells percentage from total MMP-9+, MMP-7+ and MMP-14+ cells (n=2).

FIGS. 7A-7B include graphs and micrographs showing proteases characterization of the I.P. model. (7A) WB with anti MMP-9 and MMP-7 antibodies of lesion samples at different time points. Proteases expression were normalized to a-tubulin expression. Quantification of the WB was done using ImageJ software (n=2-3). (7B) Representative images of diaminobenzidine (DAB) histochemical stain at day 10 and 14.

FIGS. 8A-8F include schemes, graphs, and an image showing that inhibition of active MMP-7 using GSM-192 mAb (anti-MMP-7) reduces endometriotic lesion weight. (8A) A schematic representation of a non-limiting experimental set up of inhibition in the S.C. model. (8B) Lesion weight in the S.C. model following MMP-7 inhibition: average weight of all lesions together per treatment (left) and average weight of lesion per animal (right). (8C) Lesion weight in the S.C. model following MMP-9 and TACE inhibition: average weight of all lesions together per treatment (left) and average weight of lesion per animal (right). (8D) A schematic representation of a non-limiting experimental set up of in-vivo inhibition in the I.P. model. (8E) Lesion weight in the I.P model following MMP-7 inhibition: average weight of all lesions together per treatment (left) and average weight of lesion per animal (right). (8F) An image of representative lesions.

FIGS. 9A-9B include a plot and a graph showing RNA-Sequencing of endometrial lesion at day 21 in GSM-192- or vehicle-treated mice in the S.C. model. (9A) Volcano plot of gene expression identified within day-21 lesions comparing between GSM-192- or vehicle-treated mice. Red and blue dots are genes that are significantly upregulated or downregulated (respectively) following GSM-192 treatment. Grey dots are not significantly differentially expressed. (9B) 1D annotation enrichment analysis done on the RNA-sequencing data. red-upregulated pathways following GSM-192 treatment; blue-down-regulated pathways following GSM-192 treatment.

FIG. 10 includes a vertical bar graph showing real time analysis of endometriotic lesions from day 14 of the I.P. model. It is shown that MMP-7 inhibition induced down regulation of angiogenesis markers.

FIGS. 11A-11B include a plot and a graph showing degradomic analysis of endometrial lesions at day 14 in GSM-192- or IgG control-treated mice in the I.P. model, highlighting potential substrates of MMP-7 activity in endometriotic lesions. (11A) Volcano plot of Log 2 protein abundance fold change comparing between GSM-192- or IgG control-treated mice. Red are substrates that are significantly upregulated in the IgG control group—potentially direct substrates of MMP-7 or down-stream substrates of MMP-7 activity. Blue are substrates that are significantly upregulated in the GSM-192 control group—potentially substrates of proteases that MMP-7 inhibits. Grey dots are not significantly differentially expressed. (11B) A graph showing Log 2 fold change (normalized to GSM-192 average) of the MMP-7 substrates: Prap 1, Lgmn, Cat, Hip1, Cfdp1, Ppia, Col7a1, Axl, Sipra, Ezr, and Tjp1, in GSM-192- or vehicle-treated mice.

FIGS. 12A-12B include a non-limiting scheme and graphs showing inhibition of active MMP-7 reduces pain-associated behavior in the I.P. model. (12A) A schematic representation of the experimental open field box test. Measuring distance was done using the EthovisionXT16 software. (12B) Normalized distance, duration, and entries to the center on days 14 and 21 post-surgery in both the GSM-treated and IgG control groups (I.P. model, n=10).

FIGS. 13A-13E include volcano plots, and graphs showing analysis of gene expression in lesions: comparing GSM-192-treated and control mice at day 14 in I.P. model and day 21 in S.C. model. (13A) A volcano plot of gene expression identified within day—14 lesions compering between GSM-192- or IgG control-treated mice. Red and blue dots are genes that are significantly upregulated or downregulated (respectively) following GSM-192 treatment. Grey dots are not significantly differentially expressed. (13B) IPA enrichment analysis done on the RNA-sequencing data from 13A. (13C) A volcano plot of gene expression identified within day—21 lesions compering between GSM-192- or vehicle-treated mice. Red and blue dots are genes that are significantly upregulated or downregulated (respectively) following GSM-192 treatment. Grey dots are not significantly differentially expressed. (13D) IPA enrichment analysis done on the RNA-sequencing data from 13C. (13E) qRT-PCR analysis of cytokine, immune cell, and angiogenic marker genes on day 14 in the I.P. model, comparing mice treated with GSM-192 and those given IgG control (n=6).

FIGS. 14A-14B include fluorescent micrographs and vertical bar graphs showing Reduction in T-cells infiltration and neo-angiogenesis following GSM-192-treatment. (14A) Representative images and quantification of CD3 staining in day 21 lesions of the I.P from mice treated with GSM or IgG control. The quantification of the covered area was done using ImageJ software (n=6, scale=200 μm). (14B) Representative images and quantification of CD34 staining in day 21 lesions of the I.P from mice treated with GSM or IgG control. The quantification of the covered area was done using ImageJ software (n=6, scale=200 μm).

FIGS. 15A-15B include a flow chart and a graph showing that degradomic analysis identifies substrates involved in the MMP-7 pathway. (15A) A flow chart showing that out of 4,436 peptides identified, 115 peptides met the significance threshold. (15B) A volcano plot illustrates neo-N-terminal peptides exhibiting decreased (red) or increased (blue) abundance following MMP-7 inhibition, annotated with the names of their parent proteins. Each point on the plot corresponds to a single neo-N-terminal peptide. Substrates potentially involved in the MMP-7 pathway are delineated within a red box.

FIGS. 16A-16B include a table and a heatmap showing that substrates associated with the MMP-7 pathway are involved in endometriosis-related pathways. (16A) A table categorizing the significant proteins, which their peptides demonstrated decreased abundance following MMP-7 inhibition, into six primary process categories related to endometriosis. This classification is based on their GO pathways, analyzed using the DAVID analysis tool. Yellow-proteins located inside the cell. Blue-proteins located in plasma membrane or extracellular space. (16B) A heatmap of the significant proteins from 16A that are known to have anti-inflammatory or anti-angiogenesis functions.

FIGS. 17A-17B include an image and bar graphs showing that MMP-7 activity leads to the degradation of Eif3e. (17A) Western blot (left) of lesions on day 21 from the I.P. model in mice treated with GSM-192 or IgG control, and a quantification graph (right) of left, as performed using ImageJ (n=5). (17B) Graphs showing quantification of dot blots of serum samples.

### DETAILED DESCRIPTION

### Compositions for Use

According to one aspect, there is provided a pharmaceutical composition comprising a therapeutically effective amount of an agent capable of inhibiting an active site of a matrix metalloprotease (MMP-7) protein, for use in the treatment of endometriosis or amelioration of at least one symptom associated therewith, in a subject in need thereof.

In some embodiments, the subject is selected as being suitable for treatment of endometriosis or amelioration of at least one symptom associated therewith, by determination of having at least one endometrial lesion characterized by increased MMP-7 expression level compared to a healthy control subject.

According to another aspect, there is provided a pharmaceutical composition comprising a therapeutically effective amount of an agent capable of inhibiting an active site of an MMP-7 protein, for use in the treatment of a subject afflicted with endometriosis or being afflicted with at least one symptom associated therewith, and determined as having at least one endometrial lesion characterized by increased MMP-7 expression level compared to a healthy control subject.

As used herein, the term “endometriosis” refers to a complex disorder associated with pelvic pain and infertility and is characterized by the implantation of endometrial tissue outside the uterus, primarily on the pelvic peritoneum and ovaries (Giudice L C, Kao L C (2004) The Lancet 364:1789-99). Endometriosis affects 6-10% of women in the general population and 35-50% of women with pain and/or infertility (Eskenazi B, Warner M L (1997) Obstet Gynecol Clin North Am 24:235-58). It is widely accepted that by retrograde menstruation (Sampson J A (1927) Am J Obstet Gynecol 14:442-469), endometrial tissue establishes itself on the peritoneum of women with endometriosis due to heritable and/or acquired defects that confer survival advantage and promote attachment, growth, neoangiogenesis, and invasion into the peritoneum.

The main clinical symptoms of endometriosis are pelvic pain, bleeding, and infertility, with the latter proposed to be related to impaired implantation due, in part, to impaired decidualization of endometrial stromal fibroblasts (ESFs). Clinical observations suggest the presence of progesterone (P4) resistance in some women with endometriosis. In addition, endometriotic lesions synthesize aromatase, a key enzyme in the biosynthesis of E2, a potential regulator of lesion growth and pain.

In some embodiments, MMP-7 expression level is determined in sample obtained or derived from the subject.

In some embodiments, a sample comprises or consists of a biopsy obtained or derived from the subject.

As used herein, the term “biological sample” includes sections of tissues such as biopsy and autopsy samples, and frozen sections taken for histologic purposes. Such samples include blood and blood fractions or products (e.g., serum, plasma, platelets, red blood cells, and the like), sputum, endometrial tissue, the uterine fundus, thyroid tissue, cultured cells, e.g., primary cultures, explants, and transformed cells, stool, urine, etc. A biological sample is typically obtained from a eukaryotic organism, such as a mammal, including a primate e.g., chimpanzee or human; cow; dog; cat; or a rodent, e.g., guinea pig, rat, mouse; rabbit.

As used herein, the term “biopsy” refers to the process of removing a tissue sample for diagnostic or prognostic evaluation, and to the tissue specimen itself. Any biopsy technique known in the art can be applied to the diagnosis and prognosis as disclosed herein, according to the method of the invention. The biopsy technique applied will depend on the tissue type to be evaluated (e.g., endometrial, etc.), the size and type of the tissue, among other factors. Representative biopsy techniques include, but are not limited to, excisional biopsy, incisional biopsy, needle biopsy, surgical biopsy, and bone marrow biopsy. An “excisional biopsy” refers to the removal of an entire endometrial tissue mass with a small margin of non-endometrial tissue surrounding it. An “incisional biopsy” refers to the removal of a wedge of endometrial tissue. Biopsy techniques are discussed, for example, in Harrison's Principles of Internal Medicine, Kasper, et al., eds., 16th ed., 2005, Chapter 70, and throughout Part V.

In some embodiments, the subject is a mammal subject. In some embodiments, the subject is a human subject. In some embodiments, the subject is a female subject. In some embodiments, the subject is a female human subject.

In some embodiments, increased expression comprises over-expression, upregulation, or both.

As used herein, the terms “overexpress”, “overexpression”, “overexpressed”, “upregulation” or “up-regulated”, are interchangeable, and refer to a protein or a nucleic acid (RNA) that is transcribed or translated at a detectably greater level, usually in an endometrial cell from a woman with endometriosis, in comparison to a cell from a woman without endometriosis. The terms include overexpression due to transcription, post transcriptional processing, translation, post-translational processing, cellular localization (e.g., organelle, cytoplasm, nucleus, cell surface), and RNA and protein stability, as compared to a cell from a woman without endometriosis. Overexpression can be detected using any conventional techniques for detecting mRNA (i.e., q-PCR, RT-PCR, PCR, hybridization) or proteins (i.e., ELISA, immunohistochemical techniques). Overexpression can be 10%, 20%, 30%, 40%, 50%, 60%, 70%, 80%, 90% or more in comparison to a cell from a woman without endometriosis. In certain instances, overexpression is at least 2-, 3-, 4-, 5-, 6-, 7-, 8-, 9-, 10-fold, or more higher levels of transcription or translation in comparison to a cell from a woman without endometriosis.

In some embodiments, MMP-7 expression level comprises mRNA expression level, protein level, or both.

In some embodiments, the pharmaceutical composition for use according to the invention is formulated for intravenous administration.

In some embodiments, the subject comprises at least one endometrial lesion. In some embodiments, the endometrial lesion is characterized by increased amount of at least one cell type selected from: an epithelial cell, a T cell, a myofibroblast, a mature blood cell, an endothelial progenitor cell, or any combination thereof, compared to a healthy control subject.

In some embodiments, any one of the: epithelial cell, T cell, mature blood cell, endothelial progenitor cell, myofibroblast, or any combination thereof, is characterized by increased expression of a gene encoding MMP-7, increased abundance of an MMP-7 protein, increased activity of an MMP-7 protein, or any combination thereof, compared to a control cell.

In some embodiments, a mature blood cell is characterized by the expression of CD31 (CD31+).

In some embodiments, an endothelial progenitor cell is characterized by expression of CD34 (CD34+).

Methods for determining expression of a gene, a protein, or both, are common and would be apparent to one of ordinary skill in the art. Non-limiting examples for methods of expression determination include, but are not limited to, RT-PCR, real-time RT-PCR, western blot, dot-blot, next generation sequencing, and others, such as exemplified herein.

In some embodiments, a subject treated according to the method of the invention and/or with the composition for use of the invention, is characterized by reduced: number of endometriotic lesions, rate of implantation of endometriotic lesions, weight of endometriotic lesions, or any combination thereof, compared to a non-treated control subject.

In some embodiments, a subject treated according to the method of the invention and/or with the composition for use of the invention, comprises at least one endometrial lesion characterized by increased expression of at least one gene associated with any one of: eosinophil migration, T cell migration, cellular defense response, myeloid dendritic cell activation, chemokine binding, lymphocyte migration, antigen processing and presentation of exogenous peptide, chemokine-mediated signaling pathway, immune response-regulating cell surface receptor signaling pathway, immunological synapse, myeloid leukocyte activation, any combination thereof, compared to a non-treated control subject.

In some embodiments, antigen processing and presentation of exogenous peptide comprises processing and presentation via MHC class II.

In some embodiments, a subject treated according to the method of the invention and/or with the composition for use of the invention, comprises at least one endometrial lesion characterized by decreased expression of at least one gene associated with any one of: epithelial cell differentiation, glycolysis and/or gluconeogenesis, glucose metabolic process, cellular carbohydrate biosynthetic process, cell-cell adherence junction, tight junction, glucan biosynthetic process, junctional membrane complex, and any combination thereof, compared to a non-treated control subject.

In some embodiments, inhibiting is by binding to an amino acid residue selected from: L181, A216, Y241, P246, Q247, N243, Y172, T180, P237, T240, H229, S101, N179, or any combination thereof, of the active site of the MMP-7 protein.

In some embodiments, inhibiting is by binding to at least one amino acid residue selected from: L181, A216, Y241, P246, Q247, N243, Y172, T180, P237, T240, H229, S101, N179, or any combination thereof, of the active site of the MMP-7 protein.

In some embodiments, at least one comprises one or more. In some embodiments, at least one comprises a plurality. As used herein, the term “plurality” refers to any integer being equal to or greater than 2.

In some embodiments, the agent comprises an antibody or an antigen-binding portion thereof.

In some embodiments, the antibody or an antigen-binding portion thereof comprises three heavy chain complementarity determining regions (CDR-H) and three light chain CDRs (CDR-L), wherein: CDR-H1 comprises the amino acid sequence: GYTFTDYN (SEQ ID NO: 1), CDR-H2 comprises the amino acid sequence: HINPNNGGTF (SEQ ID NO: 2), CDR-H3 comprises the amino acid sequence: GGGLRRGP (SEQ ID NO: 3), CDR-L1 comprises the amino acid sequence: ASESFDSYGNTFVH (SEQ ID NO: 4), CDR-L2 comprises the amino acid sequence: LVSNLE (SEQ ID NO: 5), and CDR-L3 comprises the amino acid sequence: QQNNEDPYT (SEQ ID NO: 6), or any functional analog thereof having at least 80%, 90%, 95%, 99% homology thereto, or any value and range therebetween. Each possibility represents a separate embodiment of the invention.

In some embodiments, the antibody or antigen-binding portion thereof comprises a heavy chain the comprising amino acid sequence: EVQLQQSGPELVKPGASVKIPCKASGYTFTDYNMDWMKQSHGKSLEWIGHINPNNGGT FYNQKFKDKATFIVDKSSNTAYMELRSLTSEDTAVYFCARGGGLRRGPFAYWGQGTLV TVS (SEQ ID NO: 7), or any functional analog thereof having at least 80%, 90%, 95%, 99% homology thereto, or any value and range therebetween. Each possibility represents a separate embodiment of the invention.

In some embodiments, the antibody or antigen-binding portion thereof comprises a light chain comprising the amino acid sequence: DIVTQSPASLAVSLGQRATISCRASESFDSYGNTFVHWYQQKPGQPPKLLIYLVSNLESG VPAGFRGRGSRTDFTLTIDPVEADDAATYYCQQNNEDPYTFGGGTKLEIKRA (SEQ ID NO: 8), or any functional analog thereof having at least 80%, 90%, 95%, 99% homology thereto, or any value and range therebetween. Each possibility represents a separate embodiment of the invention.

Methods for producing and/or characterizing the antibody or antigen-binding portion thereof, being disclosed herein, are elaborated in WO2020161724A1, which is incorporated herein by reference in its entirety.

As used herein, the term “antibody” refers to a polypeptide or group of polypeptides that include at least one binding domain that is formed from the folding of polypeptide chains having three-dimensional binding spaces with internal surface shapes and charge distributions complementary to the features of an antigenic determinant of an antigen. An antibody typically has a tetrameric form, comprising two identical pairs of polypeptide chains, each pair having one “light” and one “heavy” chain. The variable regions of each light/heavy chain pair form an antibody binding site. An antibody may be oligoclonal, polyclonal, monoclonal, chimeric, camelid, CDR-grafted, multi-specific, bi-specific, catalytic, humanized, fully human, anti-idiotypic and antibodies that can be labeled in soluble or bound form as well as fragments, including epitope-binding fragments, variants, or derivatives thereof, either alone or in combination with other amino acid sequences. An antibody may be from any species. The term antibody also includes binding fragments, including, but not limited to Fv, Fab, Fab′, F(ab′) 2 single stranded antibody (svFC), dimeric variable region (Diabody) and disulfide-linked variable region (dsFv). In particular, antibodies include immunoglobulin molecules and immunologically active fragments of immunoglobulin molecules, i.e., molecules that contain an antigen binding site. Antibody fragments may or may not be fused to another immunoglobulin domain including but not limited to, an Fc region or fragment thereof. The skilled artisan will further appreciate that other fusion products may be generated including but not limited to, scFv-Fc fusions, variable region (e.g., VL and VH)~Fc fusions and scFv-scFv-Fc fusions. Immunoglobulin molecules can be of any type (e.g., IgG, IgE, IgM, IgD, IgA and IgY), class (e.g., IgG1, IgG2, IgG3, IgG4, IgA1 and IgA2) or subclass.

In some embodiments, the antibody, or an antigen-binding portion thereof is selected from: Fv, Fab, F(ab′)2, scFv, or a scFv2 fragment.

In some embodiments, the antibody or antigen-binding portion thereof has specific binding affinity to the matrix metalloproteinase 7 (MMP-7) protein/enzyme (Accession number P09237).

In some embodiments, MMP-7 is Matrilysin.

In some embodiments, the antibody or antigen-binding portion thereof of the invention has specific binding affinity to the MMP-7 enzyme.

As used herein, the term “MMP-7 enzyme” refers to the enzyme form of the MMP-7 zymogen. In some embodiments, an MMP-7 hydrolyzed by a specific protease is bound by the antibody of the invention. In some embodiments, the antibody of the invention has increased binding affinity to the catalytically active MMP-7 enzyme compared to the zymogen MMP-7, inactive MMP-7, catalytically inactive MMP-7, or any combination thereof.

In some embodiments, MMP-7 comprises the amino acid sequence:

(SEQ ID NO: 9)
MRLTVLCAVCLLPGSLALPLPQEAGGMSELQWEQAQDYLKRFYLYDSETK
NANSLEAKLKEMQKFFGLPITGMLNSRVIEIMQKPRCGVPDVAEYSLFPN
SPKWTSKVVTYRIVSYTRDLPHITVDRLVSKALNMWGKEIPLHFRKVVWG
TADIMIGFARGAHGDSYPFDGPGNTLAHAFAPGTGLGGDAHFDEDERWTD
GSSLGINFLYAATHELGHSLGMGHSSDPNAVMYPTYGNGDPQNFKLSQDD
IKGIQKLYGKRSNSRKK.

In some embodiments, inhibiting is by binding to an amino acid residue selected from: L176, A211, Y236, P241, Q242, N238, Y167, T175, P232, T235, H224, S96, N174, or any combination thereof, of the active site of SEQ ID NO: 9.

In some embodiments, inhibiting is by binding to at least one amino acid residue selected from: L176, A211, Y236, P241, Q242, N238, Y167, T175, P232, T235, H224, S96, N174, or any combination thereof, of the active site of SEQ ID NO: 9.

In some embodiments, the antibody or antigen-binding portion thereof has increased binding affinity to a Zn-tripod. In some embodiments, the antibody or antigen-binding portion thereof has increased binding to affinity a Zn-tripod of MMP-7. In some embodiments, the antibody or antigen-binding portion thereof is a Zn-tripod binding antibody.

In some embodiments, the antibody or an antigen-binding portion thereof is a humanized monoclonal antibody.

It will be appreciated that for human therapy or diagnostics, humanized antibodies are preferably used. Humanized forms of non-human (e.g., murine) antibodies are chimeric molecules of immunoglobulins, immunoglobulin chains or fragments thereof (such as Fv, Fab, Fab′, F(ab′) 2 or other antigen-binding subsequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will include at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for determining binding affinity and/or association of two proteins, e.g., an antibody of the invention and MMP-7, are common and would be apparent to one of ordinary skill in the art of biochemistry. A Non-limiting example for a method of determining association includes, but is not limited to, enzyme linked immunosorbent assay (ELISA).

In some embodiments, the pharmaceutical composition further comprises pharmaceutically acceptable carrier.

As used herein, the term “carrier”, “excipient”, or “adjuvant” refers to any component of a pharmaceutical composition that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers, and diluents useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles which increase the half-life of the peptides or polypeptides in serum. Liposomes include emulsions, foams, micelles, insoluble monolayers, liquid crystals, phospholipid dispersions, lamellar layers, and the like. Liposomes for use with the presently described peptides are formed from standard vesicle-forming lipids which generally include neutral and negatively charged phospholipids and sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

The carrier may comprise, in total, from about 0.1% to about 99.99999% by weight of the pharmaceutical compositions presented herein.

As used herein, “reduce”, “reducing”, “inhibits” or “inhibiting” is by at least: 5%, 10%, 20%, 35%, 50%, 100%, 250%, 300%, 500%, 750%, or 1,000%, or any value and range therebetween. Each possibility represents a separate embodiment of the invention. In some embodiments, “reduce”, “reducing”, “inhibits” or “inhibiting” is by: 1-20%, 15-35%, 25-100%, 75-250%, 200-500%, 350-750%, or 700-1,200%. Each possibility represents a separate embodiment of the invention.

In some embodiments, a control comprises a healthy subject. In some embodiments, a control comprises a subject not afflicted with inflammation. In some embodiments, a control comprises a subject not afflicted with endometriosis. In some embodiments, a control comprises a subject having normal or baseline MMP-7 levels. In some embodiments, a control comprises a sample obtained or derived from a subject not afflicted with inflammation. In some embodiments, a control comprises a sample obtained or derived from a subject not afflicted with endometriosis. In some embodiments, a control comprises a sample obtained or derived from a subject having normal or baseline MMP-7 levels.

### Methods of Treatment

According to another aspect, there is provided a method of diagnosing endometriosis in a subject, the method comprising contacting a sample obtained or derived from the subject with the antibody described herein and determining MMP-7 expression compared to a control sample.

In some embodiments, an increased expression of MMP-7 in the sample compared to a control sample, is indicative of the subject being afflicted with endometriosis, thereby diagnosing endometriosis in the subject.

In some embodiments, an equivalent or decreased expression of MMP-7 in the sample compared to a control sample, is indicative of the subject not being afflicted with endometriosis.

In some embodiments, an increased expression of MMP-7 in the sample compared to a control sample, is indicative of the subject being suitable for treatment using an antibody or an antigen binding portion as described herein.

In some embodiments, an equivalent or decreased expression of MMP-7 in the sample compared to a control sample, is indicative of the subject being unsuitable for treatment using an antibody or an antigen binding portion as described herein.

According to another aspect, there is provided a method for treating endometriosis in a subject in need thereof.

In some embodiments, the method comprises: (a) determining expression of MMP-7 in the sample obtained or derived from the subject, wherein increased expression of MMP-7 in the sample compared to a control sample, is indicative of the subject being afflicted with endometriosis, being suitable for treatment, or both; and (b) administering to the subject determined as being afflicted with endometriosis, being suitable for treatment, or both a therapeutically effective amount of a pharmaceutical composition comprising an agent capable of inhibiting an active site of MMP-7.

In some embodiments, the agent comprises an antibody or an antigen binding portion thereof, as described herein.

In some embodiments, the sample comprises at least one endometrial tissue or fragment thereof, of the subject. In some embodiments, the sample comprises a biopsy. In some embodiments, the biopsy comprises an endometrial biopsy. In some embodiments, the biopsy comprises at least one endometrial tissue or fragment thereof, of the subject.

In some embodiments, the endometrial tissue or fragment thereof comprises at least one endometrial lesion.

In some embodiments, the endometrial tissue or fragment thereof is or comprises an ectopic endometrial tissue. In some embodiments, the sample is devoid of eutopic endometrium. In some embodiments, the endometrial tissue or fragment thereof is devoid of eutopic endometrium.

According to another aspect, there is provided a method for treating endometriosis in a subject in need thereof, the method comprising administering to the subject a therapeutically effective amount of an antibody comprising an antigen recognition region which binds a catalytic site of MMP-7, wherein the antibody inhibits the activity of MMP-7. In some embodiments, the Ki of the antibody towards MMP-7 is at least 2, 3, 4, 5 times, or any value and range therebetween, lower than a Ki of the antibody towards any one of: MMP-2, MMP-9, MMP-14, and TACE. Each possibility represents a separate embodiment of the invention.

In some embodiments, administering comprises at least once a week, at least twice a week (i.e., biweekly), at least 3 times a week, or at least 4 times a week, or any value and range therebetween. Each possibility represents a separate embodiment of the invention. In some embodiments, administering comprises 1 to 2 times a week, 1 to 3 times a week, 1 to 4 times a week, 2 to 3 times a week, 2 to 4 times a week, or 3 to 4 times a week. Each possibility represents a separate embodiment of the invention.

In some embodiments, the method comprises a step preceding the administering, comprising selecting a subject in need of and/or being suitable for treatment.

In some embodiments, the selecting comprises: (i) providing a sample of bodily fluid or a biopsy from the subject; and (ii) contacting the bodily fluid or the biopsy with the antibody described herein or an antigen-binding fragment thereof; and determining the levels of active MMP-7 enzyme in the bodily fluid or biopsy, wherein increased level of active MMP-7 compared to a pre-determined threshold, is indicative of the subject being in need of and/or suitable for treatment, as disclosed herein.

In some embodiments, an equivalent or decreased level of active MMP-7 compared to a pre-determined threshold, is indicative of the subject being not in need of and/or unsuitable for treatment, as disclosed herein.

In some embodiments, treating comprises reducing implantation rate of at least one endometrial lesion. In some embodiments, treating comprises reducing the number of ectopic endometrial lesions undergoing implantation or being implanted.

In some embodiments, treating comprises reducing or inhibiting endometriosis associated pain in the subject. In some embodiments, treating comprises reducing migration of at least one cell of an endometrial lesion.

In some embodiments, treating comprises reducing and/or inhibiting expression of at least one gene selected from: Prap1, Lgmn, Cat, Alox15, Hip1, Cfdp1, Ppia, Clo7a1, Axl, Sirpa, Ezr, Tjp, C3, samhd1, Gpb4, Akap12, Aimp1, Atp5f1a, Eif3e, or any combination thereof.

In some embodiments, treating comprises increasing expression of at least one gene selected from: Prap1, Lgmn, Cat, Alox15, Hip1, Cfdp1, Ppia, Clo7a1, Axl, Sirpa, Ezr, Tjp, C3, samhd1, Gpb4, Akap12, Aimp1, Atp5f1a, Eif3e, or any combination thereof.

In some embodiments, the method comprises determining in a sample obtained or derived from the subject the expression level of at one or more genes selected from: Prap1, Lgmn, Cat, Alox15, Hip1, Cfdp1, Ppia, Clo7a1, Axl, Sirpa, Ezr, Tjp, C3, samhd1, Gpb4, Akap12, Aimp1, Atp5f1a, Eif3e or any combination thereof. In some embodiments, reduction in the expression level of at one or more genes selected from: Prap1, Lgmn, Cat, Alox15, Hip1, Cfdp1, Ppia, Clo7a1, Axl, Sirpa, Ezr, Tjp, C3, samhd1, Gpb4, Akap12, Aimp1, Atp5f1a, Eif3e or any combination thereof, compared to a control, is indicative of the subject being responsive to the treatment disclosed herein. In some embodiments, increased or equivalent expression level of at one or more genes selected from: Prap1, Lgmn, Cat, Alox15, Hip1, Cfdp1, Ppia, Clo7a1, Axl, Sirpa, Ezr, Tjp, C3, samhd1, Gpb4, Akap12, Aimp1, Atp5f1a, Eif3e or any combination thereof, is indicative of the subject being unresponsive or non-responsive to the treatment disclosed herein.

In some embodiments, increased or equivalent expression level of at one or more genes selected from: Prap1, Lgmn, Cat, Alox15, Hip1, Cfdp1, Ppia, Clo7a1, Axl, Sirpa, Ezr, Tjp, C3, samhd1, Gpb4, Akap12, Aimp1, Atp5f1a, Eif3e or any combination thereof, compared to a control, is indicative of the subject being responsive to the treatment disclosed herein. In some embodiments, reduced expression level of at one or more genes selected from: Prap1, Lgmn, Cat, Alox15, Hip1, Cfdp1, Ppia, Clo7a1, Axl, Sirpa, Ezr, Tjp, C3, samhd1, Gpb4, Akap12, Aimp1, Atp5f1a, Eif3e or any combination thereof, is indicative of the subject being unresponsive or non-responsive to the treatment disclosed herein.

In some embodiments, the method comprises determining in a sample obtained or derived from the subject the expression level of at one or more genes selected from: Aldoa, Pcx, Gys1, or any combination thereof. In some embodiments, reduction in the expression level of at one or more genes selected from: Aldoa, Pcx, Gys1, or any combination thereof, compared to a control, is indicative of the subject being responsive to the treatment disclosed herein. In some embodiments, increased or equivalent expression level of at one or more genes selected from: Aldoa, Pcx, Gys1, or any combination thereof, compared to a control, is indicative of the subject being unresponsive or non-responsive to the treatment disclosed herein.

In some embodiments, treating comprises reducing and/or inhibiting expression of at least one metabolism related gene in at least one endometrial lesion of the subject. In some embodiments, the at least one metabolism related gene is selected from: Aldoa, Pcx, Gys1, or any combination thereof.

In some embodiments, the method comprises determining in a sample obtained or derived from the subject the expression level of at one or more genes selected from: Aldoa, Pcx, Gys1, or any combination thereof. In some embodiments, reduction in the expression level of at one or more genes selected from: Aldoa, Pcx, Gys1, or any combination thereof, compared to a control, is indicative of the subject being responsive to the treatment disclosed herein. In some embodiments, increased or equivalent expression level of at one or more genes selected from: Aldoa, Pcx, Gys1, or any combination thereof, compared to a control, is indicative of the subject being unresponsive or non-responsive to the treatment disclosed herein.

In some embodiments, treating comprises reducing and/or inhibiting expression of at least one adhesion related gene in at least one endometrial lesion of the subject. In some embodiments, the at least one adhesion related gene is selected from: Epcam, igsf5, Cldn7, Jup, or any combination thereof.

In some embodiments, the method comprises determining in a sample obtained or derived from the subject the expression level of at one or more genes selected from: Epcam, igsf5, Cldn7, Jup, or any combination thereof. In some embodiments, reduction in the expression level of at one or more genes selected from: Epcam, igsf5, Cldn7, Jup, or any combination thereof, compared to a control, is indicative of the subject being responsive to the treatment disclosed herein. In some embodiments, increased or equivalent expression level of at one or more genes selected from: Epcam, igsf5, Cldn7, Jup, or any combination thereof, compared to a control, is indicative of the subject being unresponsive or non-responsive to the treatment disclosed herein.

In some embodiments, treating comprises increasing expression of at least one immunity related gene in at least one endometrial lesion of the subject. In some embodiments, the at least one immunity related gene is selected from: Cxcl10, Stx11, il-15, Itga11, or any combination thereof.

In some embodiments, the method comprises determining in a sample obtained or derived from the subject the expression level of at one or more genes selected from: Cxcl10, Stx11, il-15, Itga11, or any combination thereof. In some embodiments, increase in the expression level of at one or more genes selected from: Cxcl10, Stx11, il-15, Itga11, or any combination thereof, compared to a control, is indicative of the subject being responsive to the treatment disclosed herein. In some embodiments, equivalent or decreased expression level of at one or more genes selected from: Cxcl10, Stx11, il-15, Itga11, or any combination thereof, compared to a control, is indicative of the subject being unresponsive or non-responsive to the treatment disclosed herein.

In some embodiments, treating comprises reducing and/or inhibiting angiogenesis in at least one endometrial lesion of the subject. In some embodiments, reduction and/or inhibition of angiogenesis is determined by expression level of one or more genes selected from: PKDCC (VLK), VEGFR2, VEGFA-120, VEGFA-164, or any combination thereof.

In some embodiments, the method comprises determining in a sample obtained or derived from the subject the expression level of at one or more genes selected from: PKDCC (VLK), VEGFR2, VEGFA-120, VEGFA-164, or any combination thereof. In some embodiments, reduction in the expression level of at one or more genes selected from: PKDCC (VLK), VEGFR2, VEGFA-120, VEGFA-164, or any combination thereof, compared to a control, is indicative of the subject being responsive to the treatment disclosed herein. In some embodiments, increased or equivalent expression level of at one or more genes selected from: PKDCC (VLK), VEGFR2, VEGFA-120, VEGFA-164, or any combination thereof, compared to a control, is indicative of the subject being unresponsive or non-responsive to the treatment disclosed herein.

In some embodiments, a control comprises mock treatment, e.g., control immunoglobulin administered. In some embodiments, a control IgG excludes an antibody or an antigen binding portion thereof, as described herein.

In some embodiments, inhibiting is with an inhibition constant (Ki) of between 10 and 20 nM, 10 and 50 nM, 10 and 100 nM, 10 and 150 nM, 10 and 175 nM, or 10 and 200 nM. Each possibility represents a separate embodiment of the invention.

In some embodiments, binding affinity is binding with a dissociation constant (KD) of 0.1 nM at most, 0.5 nM at most, 1 nM at most, 5 nM at most, 7.5 nM at most, 10 nM at most, 15 nM at most, 20 nM at most, 25 nM at most, 30 nM at most, 35 nM at most, 40 nM at most, 45 nM at most, or 60 nM at most, or any value and range therebetween. Each possibility represents a separate embodiment of the invention.

In some embodiments, binding affinity is binding with a KD of 0.1 to 50 mM, 0.1 to 1 nM, 0.5 to 5 nM, 1 to 10 nM, 7 to 15 nM, 12 to 25 nM, 17 to 35 nM, 20 to 45 nM, 32 to 55 nM, 45 to 65 nM, or 40 to 70 nM. Each possibility represents a separate embodiment of the invention.

### General

As used herein, the terms “treatment” or “treating” of a disease, disorder, or condition encompasses alleviation of at least one symptom thereof, a reduction in the severity thereof, or inhibition of the progression thereof. Treatment need not mean that the disease, disorder, or condition is totally cured. To be an effective treatment, a useful composition herein needs only to reduce the severity of a disease, disorder, or condition, reduce the severity of symptoms associated therewith, or provide improvement to a patient or subject's quality of life.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

As used herein, the term “about” when combined with a value refers to plus and minus 10% of the reference value. For example, a length of about 1,000 nanometers (nm) refers to a length of 1,000 nm±100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein, and the laboratory procedures utilized in the present invention include molecular, biochemical, bioengineering, bioprocessing, microbiological, and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells-A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Selected Methods in Cellular Immunology”, W. H. Freeman and Co., New York (1980); Molecular Cell Biology Berk A. et al. 8th edition; Molecular Biotechnology: Principles and Applications of Recombinant DN, Glick BR. 5th edition; Culture of Animal Cells: A Manual of Basic Technique and Specialized Applications Freshney IR, 7th edition; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, CA (1990); Marshak et al., “Strategies for Protein Purification and Characterization-A Laboratory Course Manual” CSHL Press (1996); all of which are incorporated by reference. Other general references are provided throughout this document.

### Materials and Methods

### Animals

Recipient wild type female mice (C57BL/6) were purchased from Envigo (Jerusalem, Israel). GFP-expressing donor female mice (C57BL/6-Tg (UBC-GFP) 30Scha/J, Jackson laboratories) were inbred at the Weizmann Institute of Science. Females were used between the ages of 8-12 weeks at the start of the experiment. All experiments and procedures were approved by the Weizmann Institute of Science Animal Care and Use Committee (IACUC approval no. 08771120-3).

### Mouse Models of Endometriosis

Endometriosis was induced using wild-type recipient mice and GFP-expressing donor mice based on previous validated models of endometriosis with a few modifications. In both models the donor females were injected subcutaneously (S.C.) 2 days prior to transplantation with 3.5 μg of 17-B-estradiol (Sigma Aldrich, dissolved in corn oil) to ensure a proliferative endometrium. Donor mice were sacrificed on the day of transplantation and their uteri were removed into a petri dish containing phosphate buffered saline (PBS) supplemented with penicillin (100 U/ml) and streptomycin (100 g/ml).

In the intra-peritoneal model (I.P.), the donors' uterine horns were sliced longitudinally to expose the endometrium and a dermal biopsy puncher was used to isolate a 2-mm uterine fragment. Recipient mice were anesthetized by an I.P. injection of a mixture of ketamine hydrochloride (60 mg/kg; Kepro, The Netherlands) and xylazine hydrochloric (10 mg/kg; VMD, Belgium) and were shaved. The abdominal cavity was opened through a midline incision and the pieces of uterine tissue were transplanted onto the inner surface of the abdominal wall using 1 μl of veterinary-grade tissue bonding agent (3M Vetbond), with their endometrial surfaces facing the peritoneum (1 piece in each side, 2 pieces total per recipient mouse). Recipient mice were given a s.c. injection of buprenorphine (0.1 mg/kg) for pain management right before surgery and in the morning after.

Abdominal layers were closed with a 6-0 absorbable suture and the skin was closed with 5-0 silk suture.

In the S.C. model, the donors' uterine horns were sliced longitudinally and cut into approximately 1 mm squares that were placed in a petri dish containing PBS supplemented with penicillin (100 U/ml) and streptomycin (100 g/ml). Recipient mice were anesthetized by a mixture of ketamine and xylazine hydrochloric and received 2 S.C. injections of 6 uterine fragments in 300 μL of PBS supplemented with antibiotics each. The S.C. injections were performed at each side of the abdomen using 1 mL syringes and 18-gauge needles.

In both models the recipient mice were injected with 3.5 μg of 17-B-estradiol once a week starting from the day of transplantation until the end of the experiment. Recipient mice were sacrificed in different time points after transplantation and lesion samples were excised and snap-frozen in liquid nitrogen or fixed with 4% paraformaldehyde, paraffin-embedded, and sectioned.

Treatment with Monoclonal Antibodies (mAb) and Pro-Domain in Endometriosis Models

In-house designed and generated inhibitors: anti-MMP-7 (GSM192), anti-MMP-9 (SDS3) and TACE pro-domain (TPD) were purified as previously described (Mohan et al, 2021; Sela-passwell et al., 2012; Wong et al., 2016).

In the S.C model, one week after transplantation, mice were injected I.P. with GSM192/SDS3/TPD or vehicle as control every day at a concentration of 3 mg/kg body weight. In the I.P. model, one day after transplantation, mice were injected I.P. with GSM192 or IgG control (In VivoMab mouse IgG1 isotype control, cat: BE0083) or vehicle as control every day at a concentration of 3 mg/kg body weight.

### Immunofluorescence Staining

Lesion samples were fixed with 4% paraformaldehyde in PBS, paraffin embedded and sectioned (4 μM). Slides were de-paraffinized and subjected to antigen retrieval in Tris-EDTA or citric acid buffer. Samples were blocked in PBS, 20% normal donkey serum, and 0.2% Triton X-100 (20 min, 25° C.) and then incubated with primary Ab in PBS, containing 2% normal donkey serum and 0.2% Triton X-100 (60 min, 25° C.). Next, samples were washed three times in PBS and incubated with a secondary antibody (60 min, 25° C.) and mounted in a mounting medium. Primary Abs: anti-GFP antibody (ab6673, Abcam), anti CD31 (ab124432, Abcam), CD34 (T-CL8927AP, Cederlane Labs).

Staining coverage was quantified by ImageJ software and presented as the mean percentage of area covered by staining out of the total lesion area. For each image a consistent binary threshold was applied, and an Analyze Particles plugin was used to detect the stained-covered area.

### Quantitative Real-Time RT-PCR

Total RNA was extracted from the lesion samples using TRIREAGENT (MRC) according to the manufacturer's protocol. RNA was reverse transcribed using High-Capacity cDNA Reverse Transcription Kit (Applied Biosystems Inc.). qRT-PCR was performed using SYBR Green PCR Master Mix (Applied Biosystems inc.) on an ABI 7300 instrument (Applied Biosystems). Values were normalized to the Actin housekeeping gene. Primer sequences are listed in Table 1 below.

Data is presented as mean fold change compared to Day 5 using the 2^{-ΔΔCT} method. The statistical analysis was done on ACT values.

TABLE 1
Primer sequences used for qRT-PCR

| Gene | Forward | Reverse |
|---|---|---|
| Mouse | ATATCGCGGTGTAAAACTCG | TAGGCTTAGGCGTTTCTGGAA |
| cd45 | TC (SEQ ID NO: 10) | (SEQ ID NO: 11) |
| Mouse | GCAGAGGCATACTTGTACCG | TGATGTTATGATGGTCCCACT |
| mmp9 | (SEQ ID NO: 12) | TG (SEQ ID NO: 13) |
| Mouse | CTGCCACTGTCCCAGGAAG | GGGAGAGTTTTCCAGTCATGG |
| mmp7 | (SEQ ID NO: 14) | (SEQ ID NO: 15) |
| Mouse | AGGCTGATTTGGCAACCATGA | CCCACCTTAGGGGTGTAATTC |
| mmp14 | (SEQ ID NO: 16) | TG (SEQ ID NO: 17) |
| Mouse | CACAAAACTTGAGAGTCGTG | GCTAGAACCCTAGAGTCAGGC |
| adam 17 | GT (SEQ ID NO: 18) | (SEQ ID NO: 19) |

### Smart-Seq2 Bulk RNA-Seq Libraries

Libraries were prepared using a modified SMART-Seq2 protocol (Picelli et al., 2014). One (1) ng of purified RNA was taken for reverse transcription with Maxima Reverse Transcriptase (Life Technologies) and whole-transcription amplification (WTA) with KAPA HotStart HIFI 2× ReadyMix (Kapa Biosystems) for 20 cycles. WTA products were purified with Ampure XP beads (Beckman Coulter), quantified with Qubit dsDNA HS Assay Kit (ThermoFisher), and assessed with a high-sensitivity DNA chip (Agilent). RNA-seq libraries were constructed from purified WTA products using Nextera XT DNA Library Preparation Kit (Illumina). The libraries were sequenced on an Illumina NextSeq 500/550. Data analysis was performed by the Nancy and Stephen Grand Israel National Center for Personalized Medicine (INCPM) in the Weizmann Institute of Science. One dimensional (1D) annotation enrichment analysis was performed on log 2foldchange of the anti MMP-7 antibody (GSM) vs. vehicle values. A false discovery rate (FDR) cutoff of 0.02 was used.

### Tissue Extraction and Western Blotting

Frozen lesion tissues were washed in PBS, homogenized in RIPA lysis buffer (EMD Millipore, Burlington, MA, USA) with a protease inhibitor (Roche, Basel, Switzerland) using a bead beater homogenizer. Then, samples were shaken at 4° C. for 10 min and centrifuged (14,000 g for 15 min at 4° C.). Supernatants were then resuspended in a sample buffer [200 mM Tris, pH 6.8, 40% glycerol, 8% sodium dodecyl sulfate (SDS), 100 mM dithiothreitol (DTT), 0.2% bromophenol blue] and boiled for 5 min. Tissue extracts were then subjected to SDS polyacrylamide gel electrophoresis (PAGE) and transferred onto polyvinylidene fluoride (PVDF) membranes by electroblotting. Membranes were blocked in PBS with Tween 20 (PBST) with 2% bovine serum albumin (BSA, 60 min, 25° C.), and then incubated with the corresponding primary Ab (Overnight, 25° C.), washed three times with PBST and incubated with a horseradish peroxidase (HRP)-conjugated secondary antibody (60 min, 25° C.). Quantification of band intensities was performed using the ImageJ analysis tool and normalized to a tubulin housekeeping gene. Abs used in this study included: MMP-9 (ab38898, Abcam), MMP-7 (ab5706, Abcam), MMP-14 (ab51074, Abcam), TACE (ab57484, Abcam), tubulin (ab4074, Abcam).

Secondary Abs (both anti-rabbit and anti-mouse) are conjugated to HRP and were purchased from Jackson ImmunoResearch (cat No. 111-001-003 and 115-001-003, respectively). Abs were used according to the manufacturer's recommended dilution.

Cell Isolation and Mass Cytometry (cyTOF)

Recipient mice were sacrificed on day 3 and day 21 after transplantation, and their lesions were harvested and pooled (lesions from 3 recipients were considered as 1 pool). Lesions were minced into small fragments and incubated in 1 ml PBS (with Mg²⁺ and Ca²⁺) containing 0.5 mg/ml collagenase type IV (Sigma-Aldrich, Rehovot, Israel) and 0.1 mg/ml DNase I (Roche) under shaking for 30 min at 37° C. The digested tissue was filtered and mashed with a syringe plunger through a 40-μm cell strainer in PBS containing PBS, and 2% fetal calf serum (FCS), to mechanically dissociate the remaining tissue. Samples were centrifuged at 400 g, and the pelleted cells were lysed for erythrocytes using a red blood cell lysis buffer (Sigma-Aldrich, Rehovot, Israel) (2 min, 25° C.). Following the above, cells were stained according to a previously published protocol (Behbehani et al., 2014). Individual mice cell suspensions were stained with 0.125 μM Cell-ID Cisplatin for viability and fixed using MaxparR Fix I Buffer. Samples were then permeabilized using Maxpar Barcode Perm Buffer and then barcoded using the Cell-ID™ 20-Plex Pd Barcoding Kit, allowing the multiplexing of samples for antigen staining. Abs used for staining are listed in Table 2 below. Before analysis, the cell suspension was incubated with Cell-ID Intercalator Iridium for 20 min. Cells were analyzed with a cyTOF2 R mass cytometer (Fluidigm). Results were normalized and de-barcoded using fluidigm cyTOF software (Zunder et al., 2015). Gating and further analysis of the CyTOF results were performed with FlowJo software (FlowJo, LLC) and cell populations were defined according to the markers listed in Table 3. tSNE (t_Distributed Stochastic Neighbor Embedding) analysis was performed using the viSNE application in the Cytobank web platform. tSNE is a machine learning algorithm used to cluster multivariate data into a two-dimensional (2D) representation.

TABLE 2
List of antibodies used in mass cytometry analysis

| | | | Isotope |
|---|---|---|---|
| Name | Product | Provider | conjugation |
| Anti-Human/Mouse/ | 3168007 | FLUIDIGM | 168Er - |
| Rat Ki-67 | | | FLUIDIGM |
| Anti-Human/Mouse | 3160012 | FLUIDIGM | 160Gd - |
| CD45R/B220 | | | FLUIDIGM |
| Anti-Mouse CD11c | 3142003 | FLUIDIGM | 142Nd - |
| | | | FLUIDIGM |
| Anti-Mouse NK1.1 | 3170002 | FLUIDIGM | 170Er - |
| | | | FLUIDIGM |
| Anti-Mouse CD4 | 3145002 | FLUIDIGM | 145Nd - |
| | | | FLUIDIGM |
| Anti-Mouse CD8a | 3146003 | FLUIDIGM | 146Nd - |
| | | | FLUIDIGM |
| Anti-Mouse TER-119 | 3154005 | FLUIDIGM | 154Sm - |
| | | | FLUIDIGM |
| Anti-Mouse Ly-6C | 3162014 | FLUIDIGM | 162Dy - |
| | | | FLUIDIGM |
| Anti-Mouse Ly-6G | 3141008B | FLUIDIGM | 141Pr - |
| | | | FLUIDIGM |
| Anti-Mouse CD64 | 3151012 | FLUIDIGM | 151Eu - |
| | | | FLUIDIGM |
| Anti- Mouse F4/80 | 3159009 | FLUIDIGM | 159Tb - |
| (BM8) | | | FLUIDIGM |
| Anti-Mouse | 3165013 | FLUIDIGM | 165Ho - |
| CD31/PECAM-1 (390) | | | FLUIDIGM |
| Anti- Mouse CD326 | 3166014 | FLUIDIGM | 166Er - |
| [EpCAM] (G8.8) | | | FLUIDIGM |
| Anti-Mouse CD45 | 3089005 | FLUIDIGM | 89Y - |
| | | | FLUIDIGM |
| Anti-mouse CD3e | BLG-100345 | Biolegend | 153Eu - |
| (maxpar ready) | | | FLUIDIGM |
| Anti-Mouse CX3CR1 | 3164023 | FLUIDIGM | 164Dy - |
| (SA011F11) | | | FLUIDIGM |
| Anti-Mouse I-A/I-E | 3209006B | FLUIDIGM | 209Bi - |
| (M5/114.15.2) | | | FLUIDIGM |
| Anti-Mouse CD11b | 3143015 | FLUIDIGM | 143Nd - |
| | | | FLUIDIGM |
| Anti-MMP9 | ab38898 | Abcam | 149Sm - Home |
| | | | made |
| Anti-MMP14 | ab51074 | Abcam | 156Gd - Home |
| | | | made |
| Anti-MMP2 | ab92536 | Abcam | 176Yb - Home |
| | | | made |
| Anti-ADAM-17 | ab57484 | Abcam | 174Yb - Home |
| | | | made |
| Anti-MMP7 | ab5706 | Abcam | 148Nd - Home |
| | | | made |
| Anti-CD34 | 14-0341-82 | Invitrogen | 152Sm - Home |
| | | | made |
| Anti-LOX | ab31238 | Abcam | 175Lu - Home |
| | | | made |
| Anti-αSMA | ab32575 | Abcam | 173Yb - Home |
| | | | made |
| Anti-MMP-13 | ab39012 | Abcam | 150Nd - Home |
| | | | made |
| Anti-Mouse GFP | | FLUIDIGM | 169Tm - |
| | | | FLUIDIGM |

TABLE 3
Gating and cell population definition in mass cytometry analysis

| ID | Markers |
|---|---|
| Epithelial cells | CD326+; CD45− |
| Endothelial progenitors | CD326−; CD45−; CD31−; CD34+ |
| Mature endothelial cells | CD326−; CD45−; CD31+ |
| T cells | CD326−; CD45+; CD11b−; CD3e+ |
| Monocytes | CD326−; CD45+; CD3e−; CD11b+; Ly-6G−; F4/80−; Ly-6Chi |
| Dendritic cells | CD326−; CD45+; CD3e−; CD11c+; MHC class II+ |
| Neutrophils | CD326−; CD45+; CD3e−; CD11b+; Ly-6G+ |
| Macrophages | CD326−; CD45+; CD3e−; CD11b+; Ly-6G−; Ly6C^{low + mid.}, |
| | F4/80+ |
| Myofibroblasts | CD326−; CD45−; CD31−; CD34−, a-SMA+ |

### Terminal Amine Isotopic Labeling of Substrates (TAILS) Degradomic Analysis

Prior to protein denaturation and labeling, proteins of samples were extracted by mechanical homogenization and sonication. This process was performed in a buffer containing 4 M Guanidine hydrochloride (Sigma-Aldrich), 250 mM HEPES (Sigma-Aldrich (pH 7.8)) and protease inhibitor cocktail (complete EDTA-free, Roche). TAILS analysis, whereby protein N-termini were enriched, was performed according to a previously described protocol, using 10plex Tandem mass Tag labeling (TMT, #90,110, Thermo Fisher) and adding the trypsin at a ratio of 1:200 (trypsin/protein ratio). Peptides were introduced into the mass spectrometer by means of Waters nanoAcquity HPLC system connected to a Symmetry trap column [180 μm×20 mm] and Analytical column HSS T3 75 μm×250 mm, both from Waters.

Data was acquired on Q exactive HF mass spectrometer (Thermo Scientific) with Top 15 method. Raw files were searched against the Mus musculus database compiled from the UniProt reference proteome using Sequest from Proteome Discoverer™ 2.4 software (Thermo). The following parameters were selected for database searches: semi-ArgC for enzyme specificity with tolerance of one missed cleavage; carbamidomethyl (C) and TMTpro (K) as fixed modifications, and acetyl (N-term), pyroQ (N-term), TMTpro (N-term), oxidation (M), deamidation (NQ), as variable modifications. Precursor mass error tolerance of 10 ppm and fragment mass error at 0.02 Da. Percolator was used for decoy control and FDR estimation (0.01 high confidence peptides, 0.05 medium confidence).

### Statistical Analysis

Data were analyzed by unpaired, two-tailed t-test to compare between two groups or by one-way ANOVA to compare several groups. After the null hypothesis was rejected (p<0.05), Tukey's Honestly Significant Difference or Dunnett tests were used for follow-up pairwise comparison of groups in the one-way ANOVA. Data are presented as mean±SEM; values of p<0.05 were considered statistically significant (*P<0.05, ** P<0.01, *** P<0.001).

### Trans-Well Migration Assay

Z-12 endometriotic cells (1×10⁵) were placed in the upper chambers of 24-well trans-well plates (BD Biosciences; 8 μm pore size) with either 1.5 μM GSM-192 (anti active MMP7 antibody) or IgG control in serum-free medium. The lower chambers were filled with medium containing 10% serum and the same antibodies (1.5 μM GSM-192 or IgG control). After incubating for 15 hours, the cells were fixed with 4% paraformaldehyde (PFA). Cells that did not migrate to the lower chamber were removed from the upper well and migrated cells were stained with 0.05% crystal violet.

### Example 1

### Establishment of Subcutaneous (S.C.) and Intraperitoneal (I.P.) In-Vivo Endometriosis Models

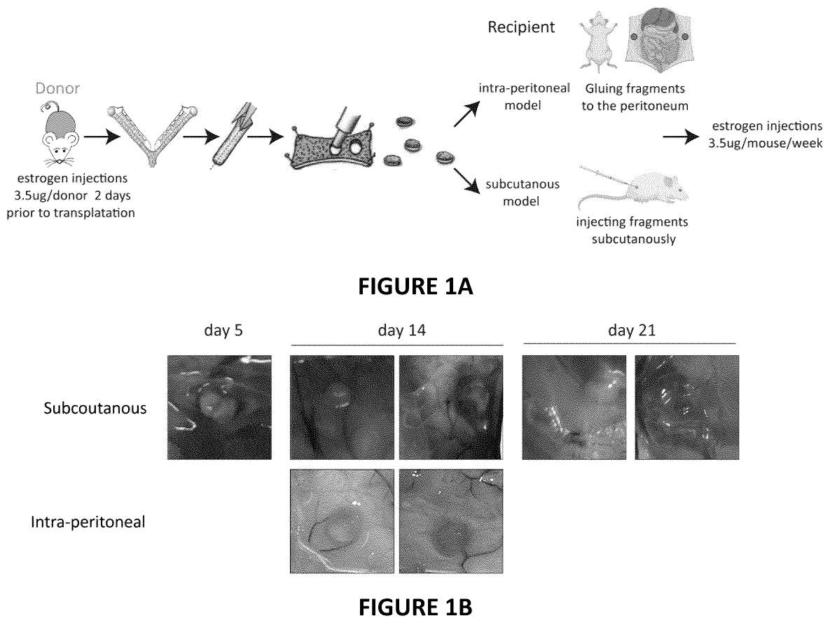

Endometriosis in women is an endo-transplantation disease in which endometrial fragments are implanted spontaneously outside the uterine cavity. Mice do not have menstrual cycles and thus do not develop this disease spontaneously. Thus, the inventors established an exo-transplantation models that resembles endometriosis using donor and recipient mice. This donor-recipient model is the leading murine model in the endometriosis research field. Briefly, donor mice (GFP-C57BL/6) were injected with 3.5 μg of estradiol and 2 days after, their uteri were excised and placed in sterile saline with antibiotics. Endometriosis was recapitulated in C57BL/6 recipient mice via subcutaneous injection (S.C. model) or intraperitoneal adhesion (I.P. model) of GFP-C57BL/6 uterus fragments. In the S.C. model, after the exposure of the endometrium, the uterus was minced to 1 mm pieces using a razor, and the fragments were then injected S.C. into recipient mice (1:3 donor: recipient ratio, 6 pieces per injection). In the I.P. model, the uterine horns were opened longitudinally with scissors to expose the endometrium and 2 mm biopsies were taken using a puncher. Then, the uterus pieces were glued to the recipient's abdominal wall with the endometrium layer facing the peritoneum (1 piece in every side). Recipient mice were administered 3.5 μg estradiol once a week from transplantation day until implant excision (FIG. 1A). Implants were excised and analyzed at different time points. In the S.C. model the uterine fragments were implanted on the outer membrane of the muscle layer surrounding the abdomen cavity called Scarpa's facia and most of the lesions adhered also to the skin from the other side. In the I.P. model, the uterine fragments were implanted on the parietal peritoneum. In the S.C model the implants phenotype is more heterogeneous as they appear at different locations, having different sizes and colors. In the I.P. model the lesions are less heterogeneous and share similar morphology (FIG. 1B).

### Example 2

### Histological Characterization of S.C and I.P Models Provide a View of Endometrial Lesion Implantation Process

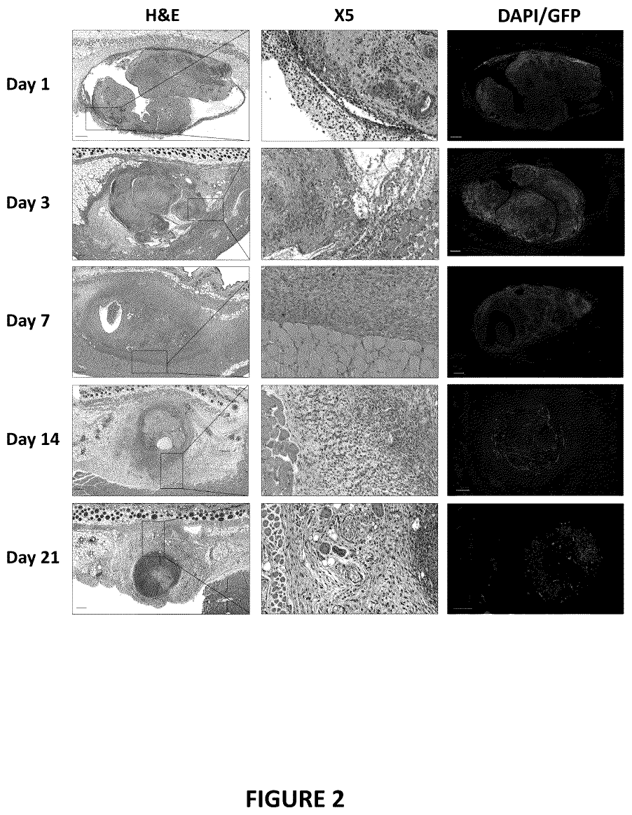

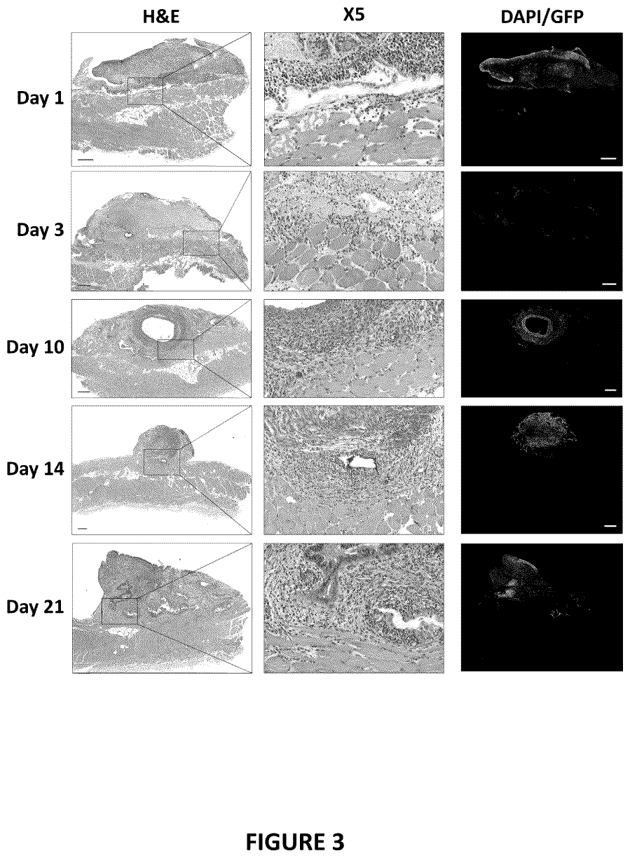

To characterize lesion apposition, adhesion and growth, lesions and their surrounding tissue were sectioned and stained with both H&E histochemical staining and immunofluorescence anti-GFP staining at different time points (FIGS. 2-3).

Initially in both models, at day 1, the borders between the endometrial fragments and the bed are very distinct (FIGS. 2-3, day 1). This is the apposition stage in which the fragments are close and touching the surface of the peritoneum (I.P) or Scarpa's facia membrane (S.C.), but they are not fully attached to it. In addition, in both models there is migration of immune cells from the host to the lesion at all-time points, starting from day 1. The S.C model only displays full attachment of the lesion to the membrane bed by day 7 (FIG. 2). At this timepoint the borders between the uterine fragments and the membrane bed and between the uterine fragments themselves are less visible with no gaps and with the host tissue and the uterine fragments invading one another. Additionally, the host cells completely surround the lesion and the inner cells of the lesion start to die, probably due to insufficient blood supply.

The I.P. model is the first to display full attachment. By day 3 the gap caused by the glue layer becomes smaller, and the lesion is fully attached to the peritoneum (FIG. 3). This attachment is accompanied by migration of more host cells into the lesion as observed by the GFP stain (more non-GFP cells inside the lesion). At this timepoint, the border between the uterine fragments and the peritoneum becomes less apparent as the endometrium and peritoneum invade one another. By day 10 the lesion is fully integrated in the host tissue (FIG. 3).

The H&E and GFP images emphasize the differences between the models, as the lesion of the S.C. model are more heterogeneous in their size and shape compared to the lesions from the I.P. model. This histological characterization provides a view of the lesion implantation process that begins with apposition, followed by adhesion and invasion. In parallel to this process there is a gradual increase in inflammation and vascularization of the uterine lesions for survival. Thus, the next step of characterization was to observe angiogenesis and inflammation time-line of both models.

### Example 3

### Characterization of Angiogenesis Time-Line in In-Vivo Models

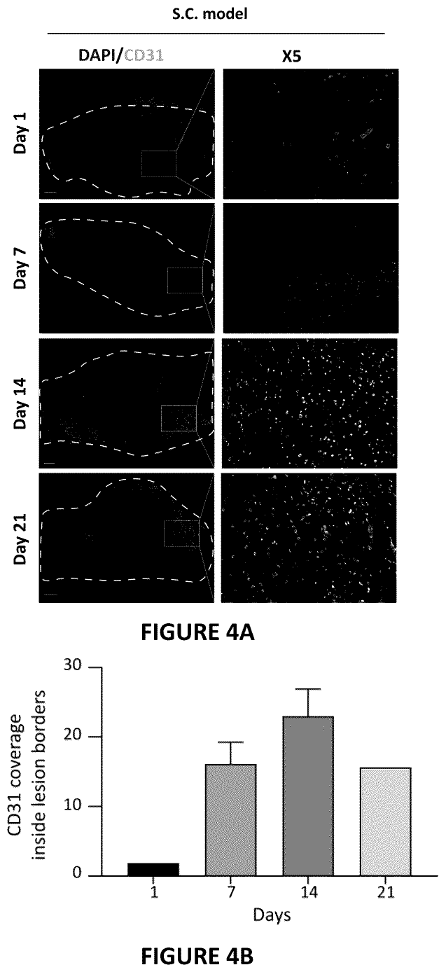

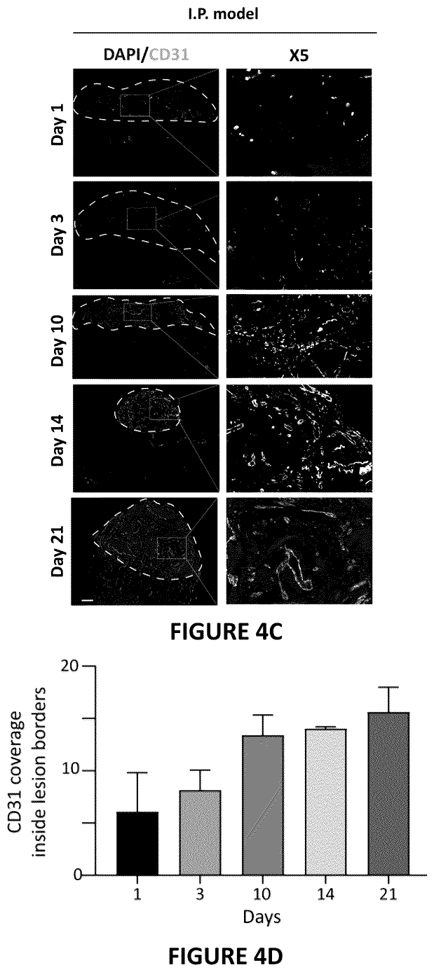

As angiogenesis is crucial for the survival of endometriotic lesions, the inventors set out to characterize the vascularization process during the stages of lesion implantation using CD31 staining, which is a marker for mature blood vessel. Vascularization was then quantified by measuring the CD31 percent covered area inside lesion borders (FIG. 4). In the S.C model, on day 1 there is a very little expression of CD31, however on day 7 more CD31 expression is observed at the border with the lesion. Between days 14-21 the levels of expression of this marker increase greatly in the lesion and the host tissue that surrounds the GFP+ center expressing CD31 (FIGS. 4A-4B). In the I.P. model, day 1 marks the lowest expression levels of CD31 in the lesion. The expression level of CD31 doubles by day 10 and remains at this level until the end of the experiment, by day 21 (FIGS. 4C-4D). The results from the CD31 staining are very similar between the models and show that the host tissue has a surprising tendency to accommodate the ectopic uterine fragments by connecting them to the vascular network and thus rescuing some of their cells from death.

### Example 4

### Characterization of Immune Cells in S.C. Model

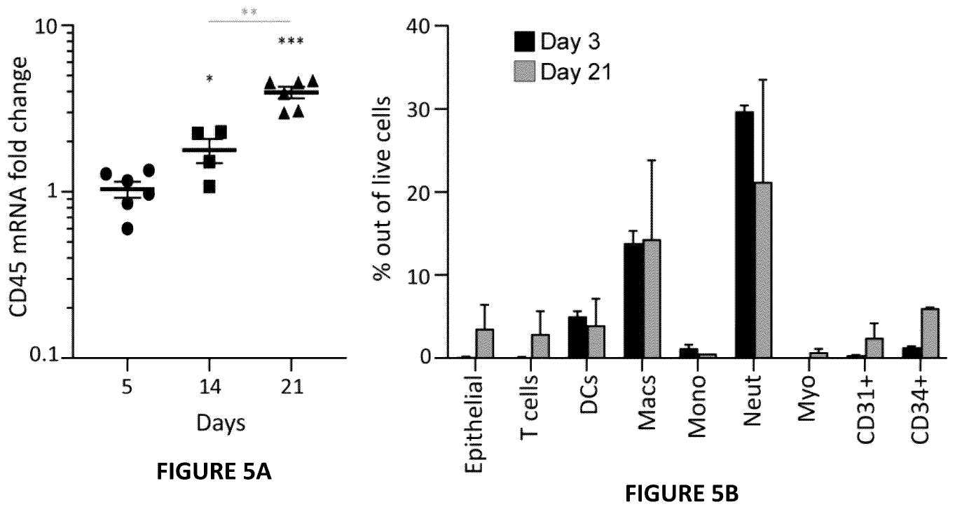

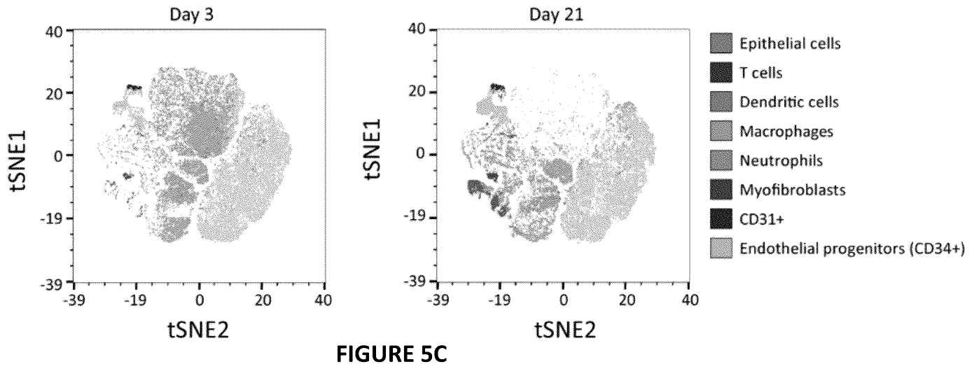

Endometriosis is a chronic inflammatory disease in which patients exhibit elevated cytokine levels and immune cells both in lesions and in the peritoneal fluid. Thus, the next step was to assess the nature of inflammation throughout the implantation process. In the S.C. model, mRNA analysis showed that the expression of general immune cell marker CD45 significantly increases throughout lesion implantation (FIG. 5A). A preliminary cyTOF experiment comparing day 3 (early lesion implantation) and day 21 (late lesion implantation) in the S.C. model showed that the general cell population composition changes during lesion implantation. There are more epithelial cells, T-cells, mature blood vessels (CD31+), and endothelial progenitors (CD34+) in late stages of implantation (day 21) compared to early stages (day 3). The increase in the number of CD31+ and CD34+ cells strengthens the conclusion of increased angiogenesis with time of implantation. On the other hand, the results demonstrate a decrease in the monocyte and neutrophil populations during lesion implantation (FIG. 5B).

Analysis of the cell populations using t-distributed stochastic neighbor embedding (tSNE) shows a general view of the cell population composition changes that occurred during lesion implantation (FIG. 5C). This tSNE analysis confirms the gating results of FIG. 5B. In addition, it shows that a big population of immune cells that contains part of the macrophages and neutrophils at day 3 is missing at day 21. The gating analysis (FIG. 5B) shows that there is not much difference between the percent of macrophages between the timepoints of the models.

Overall, the increase in T cells infiltrating the lesion together with the decrease in neutrophils occurring between day 3 and day 21 indicate that there is a transition between an acute myeloid inflammation to a chronic lymphatic inflammation.

### Example 5

### MMP-7 Activity Elevates During Lesion Implantation and Vascularization in Both In-Vivo Models

Protease activity and extracellular matrix (ECM) remodeling play key roles in implantation processes. However, their specific role in endometriosis is still poorly understood. Thus, the next level of model characterization was to examine in both models the expression of different ECM proteases known to be important in the progression of endometriosis.

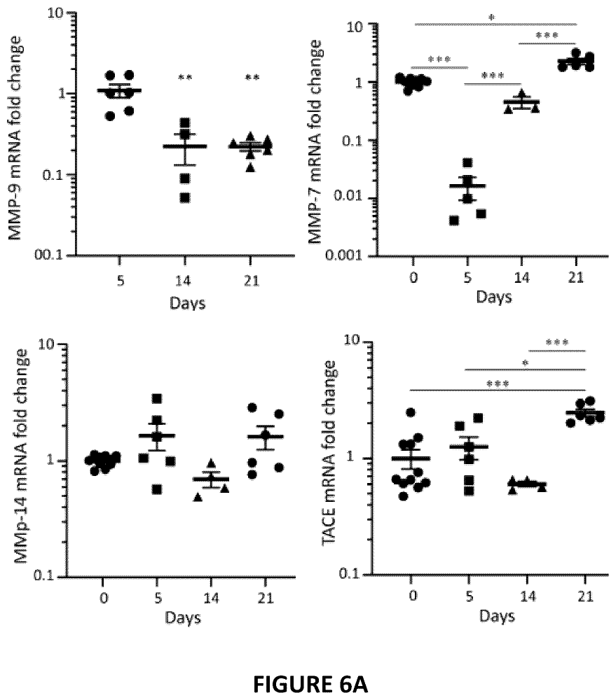

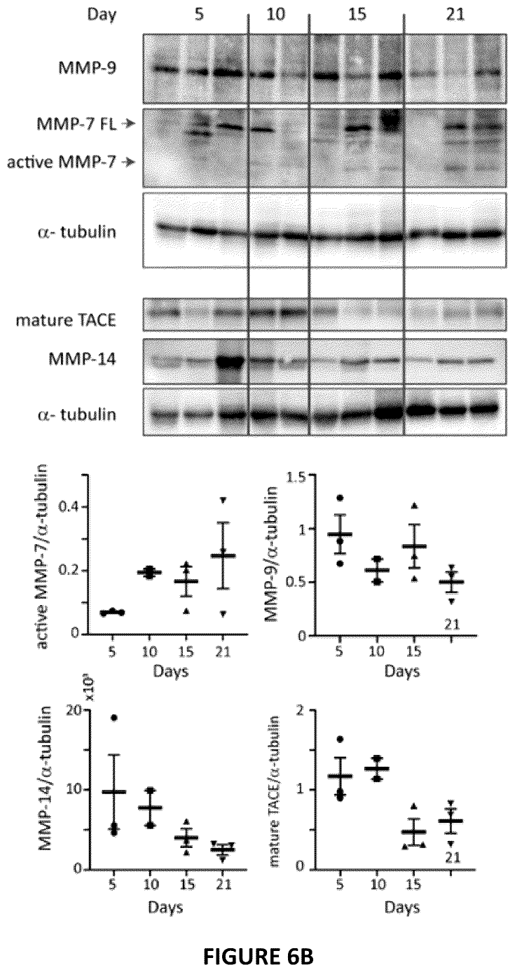

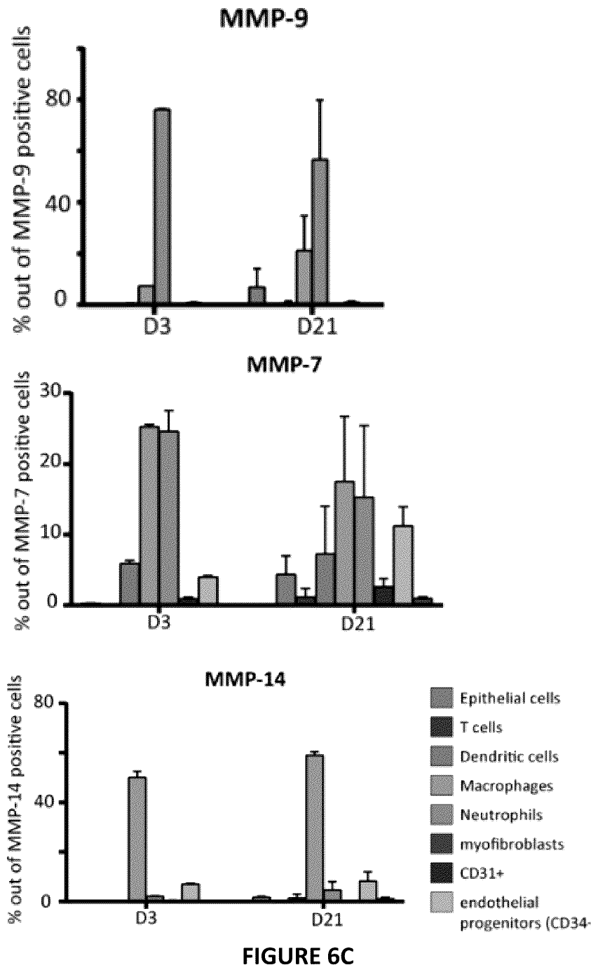

First, the inventors examined the expression levels of different ECM proteases in the S.C. model in different time points during lesion implantation: MMP-9, MMP-7, ADAM-17 (TACE), and MT1-MMP. MMP-9 is a known mediator of inflammation and plays a role in many inflammatory conditions, like endometriosis. Thus, the inventors characterized the expression of this enzyme throughout the S.C model. mRNA and western blot analyses showed that the expression of MMP-9 decreases over time of lesion implantation in this model in parallel to the decrease in myeloid cells (FIGS. 6A-6B).

MMP-7 is a known mediator for angiogenesis in various cancers and studies have shown its potential role in the progression of endometriosis (Bruner-Tran et al., 2002; Matsuzaki et al., 2010; Chatterjee et al., 2018). mRNA analysis showed a significant increase in the expression of MMP-7 in later stages of lesion implantation in the S.C. model (Day 14 and 21) (FIG. 6A). Western blot analysis in this model demonstrates no change in the expression of the full-length enzyme during different stages of lesion implantation. Nonetheless, it is shown that there is an increase in the levels of the active form of MMP-7 over time (FIG. 6B).

An additional ECM protease that the inventors have examined in the S.C. model was ADAM17 (TACE), which was shown before to play a role in the invasion of human endometriotic cells (González-Foruria et al., 2017; Miller et al., 2013). mRNA expression of TACE in the S.C. model was higher at day 21 compared to day 5 and 14 (FIG. 6A). However, western-blot analysis of the mature TACE (the active form) showed a decrease over time of implantation (FIG. 6B).

Another MMP that was shown to be elevated in association with endometriosis in woman is MT1-MMP (MMP-14) (Chung et al., 2002; Ueda et al., 2002). mRNA and protein analysis of MT1-MMP in the S.C. model showed that while it is expressed throughout lesion implantation, its expression levels do not change at different stages of implantation (mRNA and protein, FIGS. 6A-6B, respectively).

In order to examine the cell origin of the observed MMPs, cyTOF experiment was performed (FIG. 6C). Macrophages and neutrophils are the main source of MMP-7 at early stages of lesion implantation (day 3). However, the endothelial progenitor cells (CD34+) appear to be an additional source of MMP-7 at later stages of implantation (day 21) (FIG. 6C). The main source of MMP-14 and MMP-9 in early and late stages of implantation are macrophages and both macrophages and neutrophils, respectively. These results are in line with the cyTOF results (FIGS. 5B-5C) showing that the neutrophil and part of the macrophage populations decrease over the time of implantation. This accounts for the decrease in the expression of MMP-9.

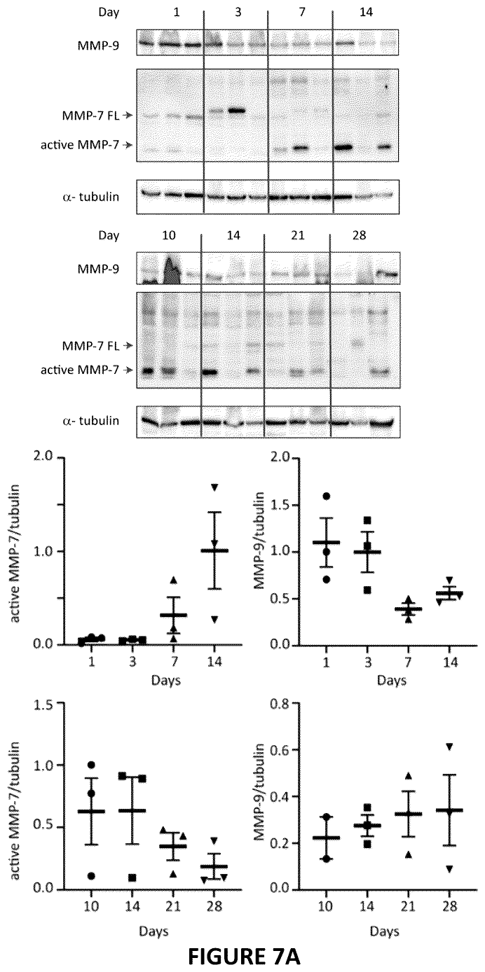

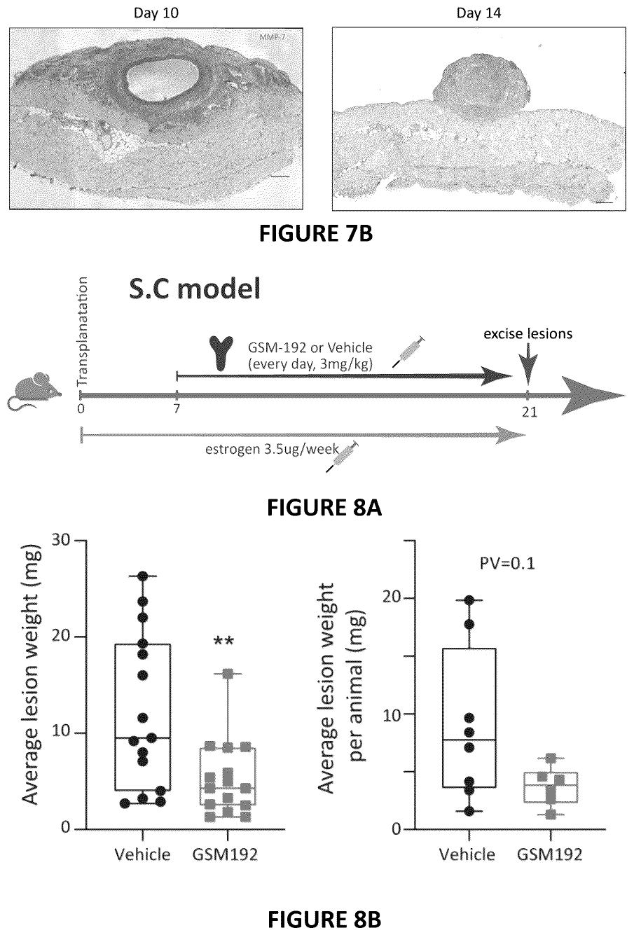

Results from the S.C. model demonstrate that MMP-7 is elevated in terms of expression and activity mainly at the later stages of lesion implantation and may serve as a key player during late stages of pathological implantation. Thus, the inventors tested the activity of this specific enzyme throughout lesion implantation in the I.P. model and found that the activity of MMP-7 is low in the beginning of lesion implantation (Day 1-3), gradually elevates through day 7 and peaks at day 10-14. However, MMP-7 activity decreases by day 21 (FIG. 7A). The staining of MMP-7 at the peak of its activity (day 10 and 14) showed that it is expressed in all parts of the lesion (FIG. 7B).

The expression of MMP-9 in the I.P. model is also similar to its expression in the S.C. model. In the I.P. model the expression of this enzyme was high in the beginning of lesion implantation (day 1-3) and was decreased from day 7 onward (FIG. 7A).

The results regarding ECM regulators in both S.C and I.P. models suggest that MMP-7 serves as a key player during a specific time window of endometriotic lesion implantation and highlights the potential of using MMP-7 inhibition to prevent endometriosis progression. Since the cell source of MMP-7 at later stages are mainly macrophages, neutrophils, and endothelial progenitor cells, the inventors hypothesize that its role is associated with lesion angiogenesis and inflammatory pathways.

### Example 6

### Inhibition of Active MMP-7 Using GSM-192 mAb Reduces Endometriotic Lesion Weight

Since MMP-7 showed a unique activity timeline during both models of lesion implantation, in which the activity increased in later stages of implantation, the inventors next step was to inhibit this enzyme, and so test whether it is a critical player during lesion implantation. For inhibition, the inventors used an in-house monoclonal antibody (mAb) termed “GSM192” which targets the active form of the enzyme (Mohan et al., 2021).

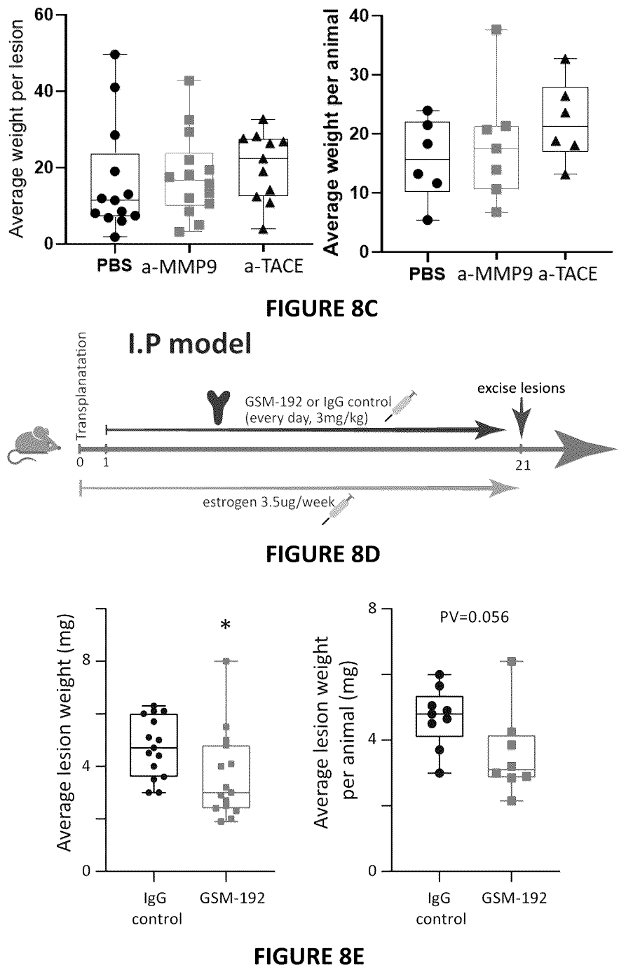

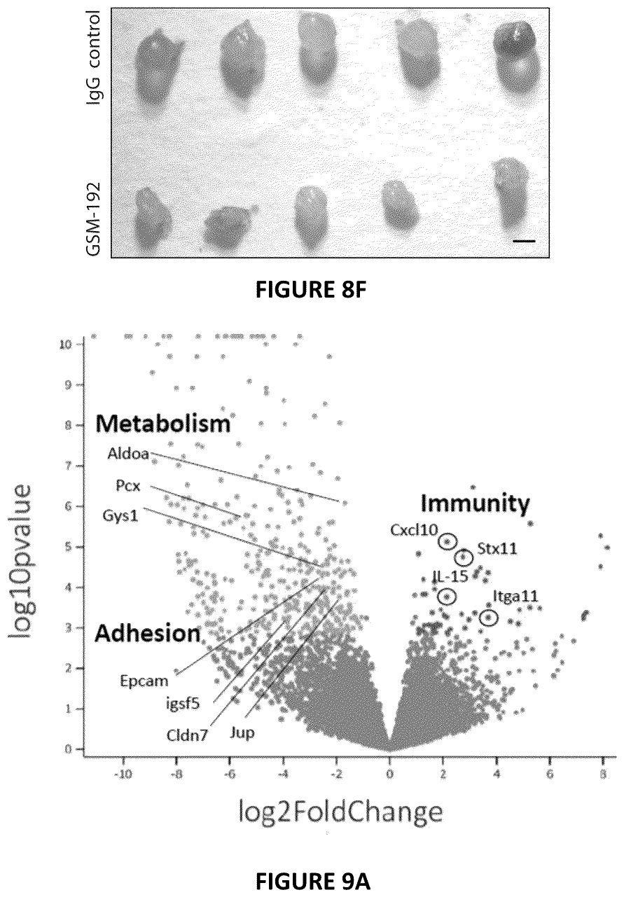

The inhibition of MMP-7 in the S.C. model started 7 days after transplantation and included intra-peritoneal injections of GSM192 (3 mg/kg) or vehicle (PBS) every day, until day 21 (FIG. 8A). Following MMP-7 inhibition in the S.C. model the average lesion weight decreased by more than 40% when calculating the average of all lesions together per treatment and more than 60% when calculating the average lesions average weights per animal (FIG. 8B). When testing other enzyme inhibitors in the S.C. model, including TACE Pro-Domain (TPD) that inhibits TACE and SDS3 mAb that inhibits MMP-9, no significant reduction in lesion weight was observed (FIG. 8C).

The inhibition of MMP-7 in the I.P model started one day after transplantation and included intra-peritoneal injections of GSM192 (3 mg/kg) or vehicle (PBS) every day. Lesions were extracted on day 21 (FIG. 8D). Following MMP-7 inhibition in the I.P. model, the average lesion weight decreased by more than 30-60% when calculating either lesions average weights per animal or all lesions together per treatment (FIG. 8E).

It is noteworthy that since lesion morphology is less heterogeneous in the I.P. model compared to the S.C. model, testing the MMP-7 inhibitors effects on lesion development was more distinct and consistent in the I.P. model. The results from the in-vivo inhibition experiments demonstrate that MMP-7 is indeed a key player in the development and implantation of endometriotic lesions.

Further, the inventor used a trans-well migration assay, wherein cells of a human endometriotic cell line (Z-12 cells) were treated with an anti MMP7 antibody (GSM-192) to show that specific inhibition of the active MMP7 significantly decreased cell migration (e.g., across the trans-well membrane), compared to a control IgG.

### Example 7

### Revealing the Mechanism Behind MMP-7 Activity in Lesion Implantation

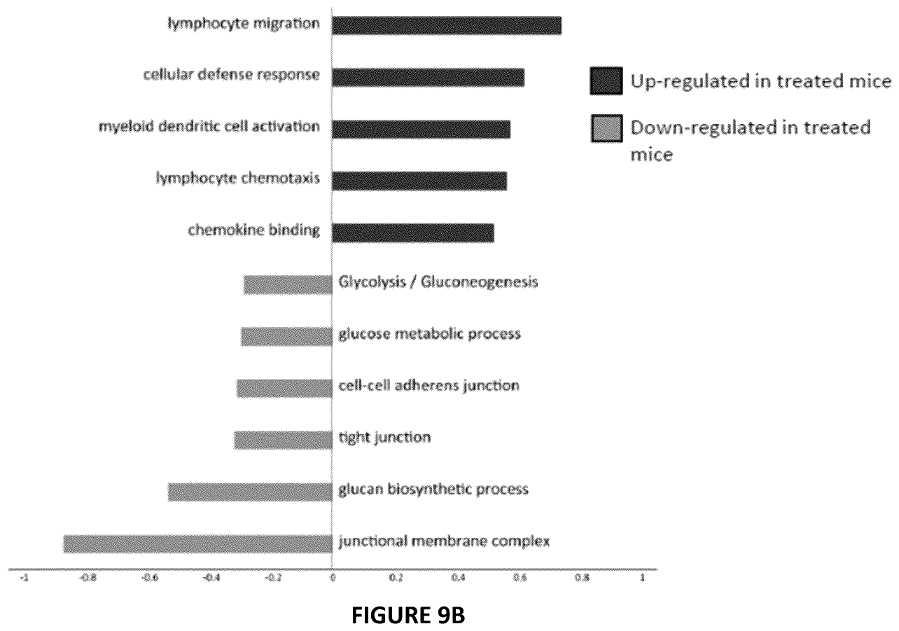

In order to discover the molecular mechanism behind MMP-7 activity in lesion implantation, the inventors performed RNA sequencing on S.C. lesions on day 21 that were treated either with GSM192 or vehicle. The RNA sequencing revealed a few differently expressed upregulated genes and a lot of differently expressed downregulated genes following MMP-7 inhibition (FIG. 9A). When performing 1D annotation enrichment analysis the inventors revealed that many inflammatory pathways were upregulated (e.g., T-cells migration, cellular defense response, myeloid activation etc.; FIG. 9B). On the other hand, pathways related to metabolism and adhesion were down regulated (e.g., glycolysis, tight junctions, cell-cell adherence etc.; FIG. 9B).

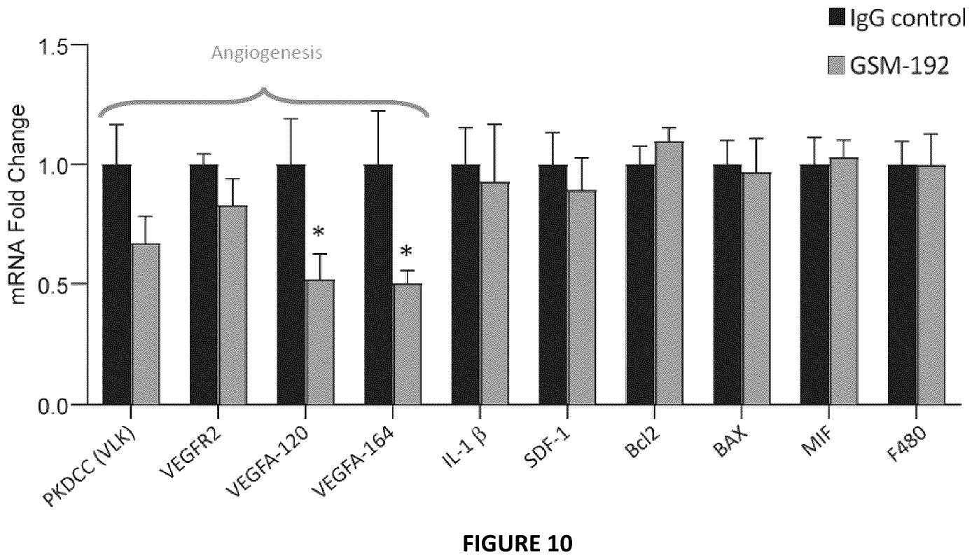

Further, the inventors have shown that in the I.P. model angiogenesis markers are down regulated following MMP-7 inhibition (FIG. 10). In this regard, the levels of genes, e.g., PKDCC (VLK), VEGFR2, VEGFA-120, and VEGFA-164, were shown to be reduced in GSM-192 injected animals, compared to control.

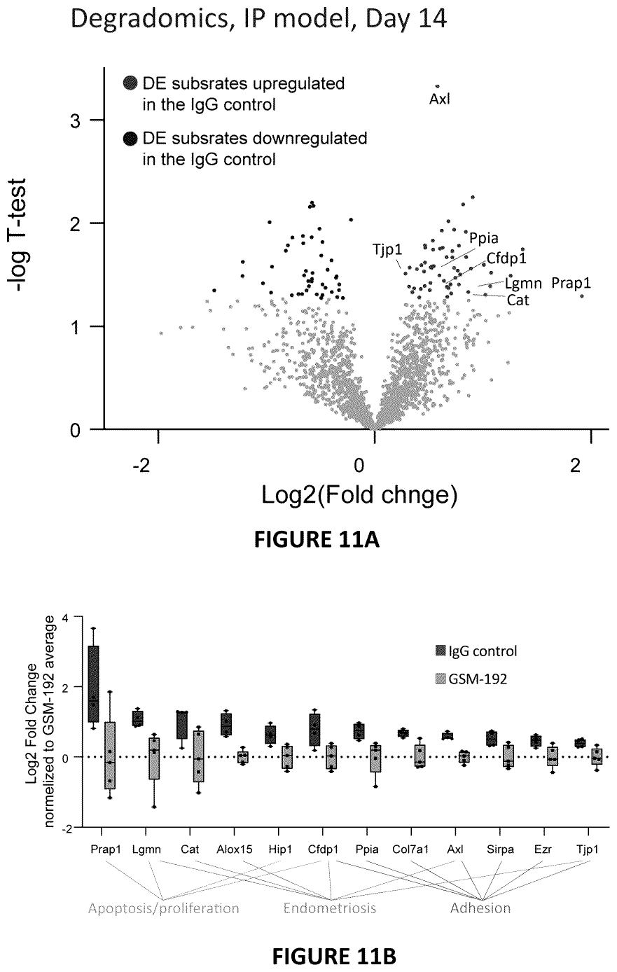

Further, the inventors have shown using a degradomic assay in the I.P. model that potential direct substrates of MMP-7 or potential down-stream substrates of MMP-7 activity in the lesion, are associated with endometriosis, adhesion, proliferation or migration. (FIG. 11). These direct substrates of MMP-7 or down-stream substrates thereof included: Prap 1, Lgmn, Cat, Hip1, Cfdp1, Ppia, Col7a1, Axl, Sipra, Ezr, and Tjp1.

### Example 8

### Inhibition of Active MMP-7 Reduces Pain-Associated Behavior

Following the discovery that MMP-7 inhibition reduces lesion sizes in mice, the inventors explored its effects on endometriosis-related pain, a primary symptom in endometriosis patients.

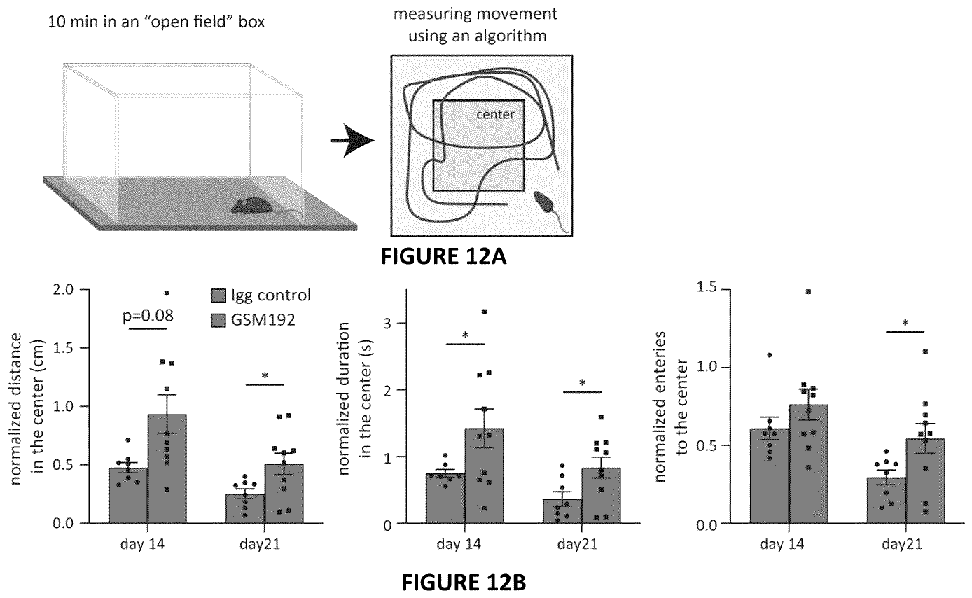

Since mice cannot communicate pain verbally, the inventors relied on behavioral tests that infer pain levels, such as the open-field test (FIG. 12A). Typically used to assess stress or anxiety, this test can also be used as a pain indicator through changes in locomotor activity. Rodents naturally explore new environments but exhibit reduced exploration and stay closer to walls when under pain states. Thus, less time in the center and more time near walls suggest higher pain levels.

Mice with endometriosis from the I.P model, treated with GSM-192, entered the center more frequently and traveled more within it by day 21, with a significant increase in center duration starting already from day 14 (FIG. 12B). This indicates that endometriotic mice treated with GSM-192 experience lower endometriosis-related pain compared to control (FIG. 12B).

### Example 9

### Inhibition of Active MMP-7 Reduces Inflammation and Angiogenesis in the Lesions

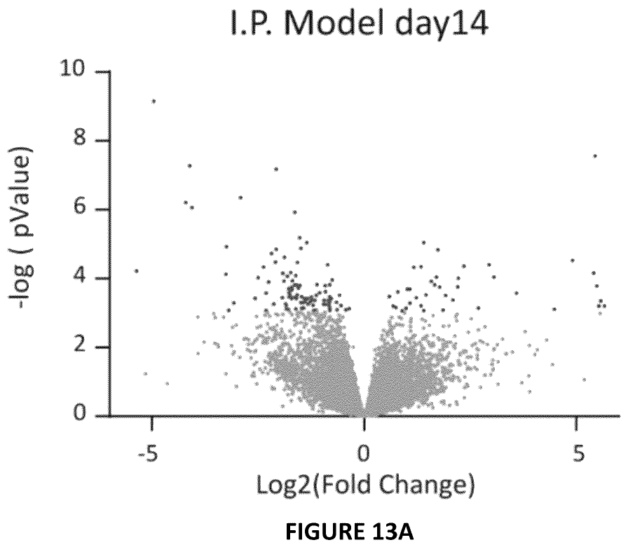

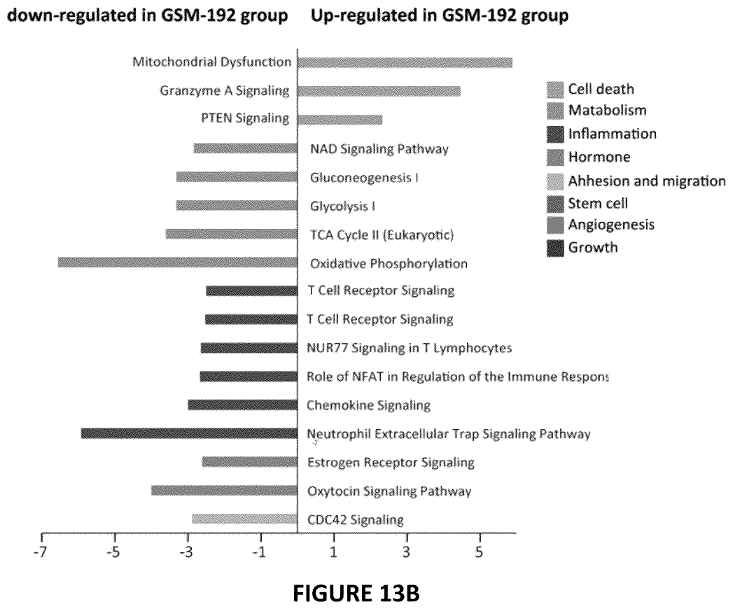

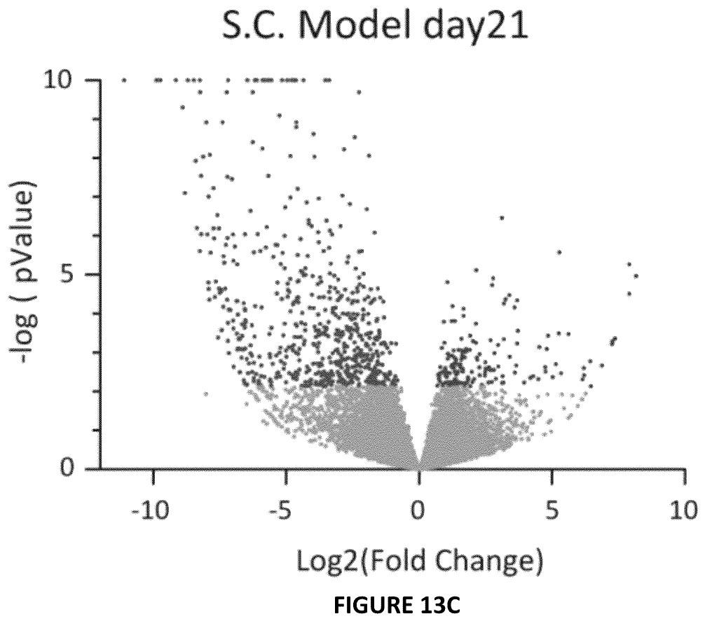

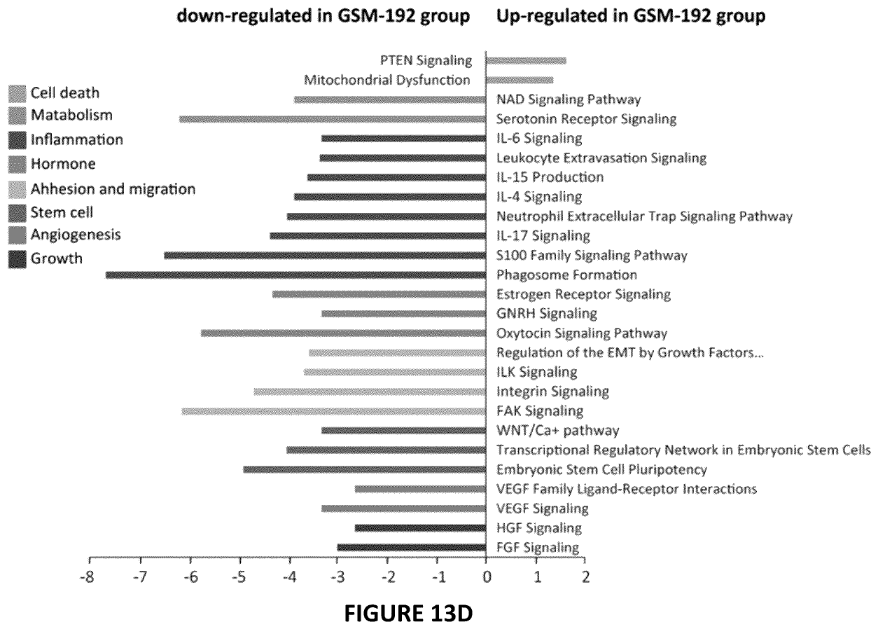

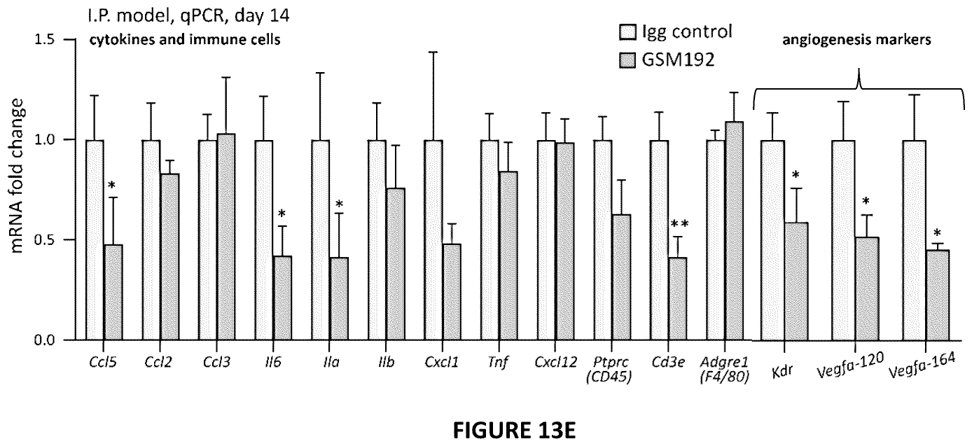

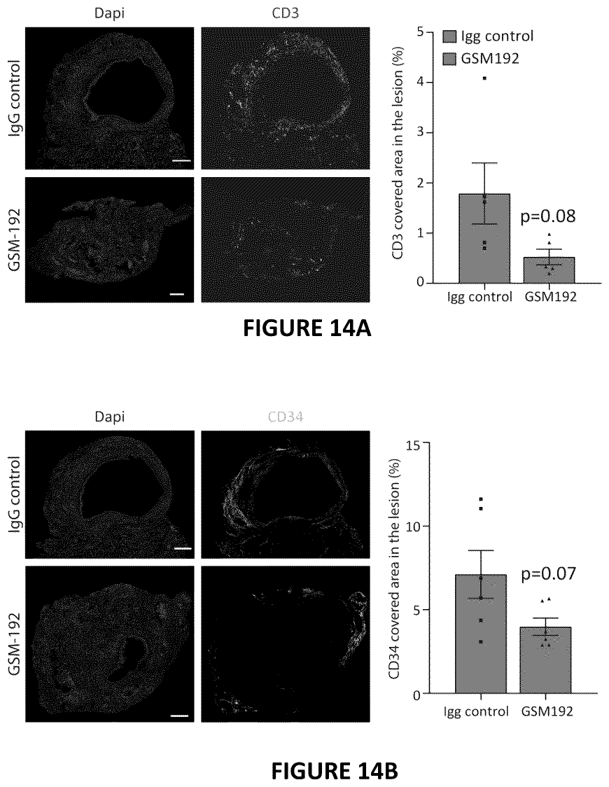

To identify the molecular pathways influenced by MMP-7 in endometriosis lesions, RNA sequencing was conducted on lesion samples from the subcutaneous (S.C.) model on day 21 and the intraperitoneal (I.P.) model on day 14 that were treated either with GSM-192 or Vehicle/IgG control. The findings from day 14 (I.P. model) are depicted in a volcano plot in FIG. 13A, illustrating that several genes were significantly upregulated (indicated in red) or downregulated (in blue) after GSM-192 treatment. Ingenuity Pathway Analysis (IPA) showed that the main pathways diminished involved inflammation, metabolism, and hormone functions, while cell death pathways increased after treatment (FIG. 13B). These findings are consistent with the RNA sequencing data from day 21 (S.C. model), where a significant number of genes were observed to be downregulated compared to a smaller number of significantly upregulated genes (FIG. 13C). Similarly, IPA demonstrated a decrease in pathways associated with inflammation, metabolism, hormone functions, and additionally, migration and angiogenesis following MMP-7 inhibition. Cell death pathways were also found to be upregulated in this analysis (FIG. 13D). Real-time analysis of RNA from lesions of the I.P. model at day 14 confirmed a decrease in inflammatory cytokines, including Il6 and Il1α, as well as a reduction in the T cell marker CD38 and the T-cell chemokine Ccl5. Additionally, the analysis indicated a decrease in angiogenic markers such as Vegfa and Kdr (FIG. 13E). Staining of lesions from the I.P. model at day 21 (one week after the RNA collection), verified a reduction in the T-cell marker (CD3e) and the marker for new blood vessels (CD34) within the lesion (FIGS. 14A-14B, respectively).

These findings of reduced angiogenesis, metabolism, and inflammation pathways, along with heightened cell death after MMP-7 inhibition, account for the observed phenotype of decreased lesion size and lowered pain levels post-treatment.

### Example 10

### Degradomic Analysis of Endometriotic Lesions from GSM-192-Treated Mice Revealed Potential Substrates of the MMP-7 Pathway

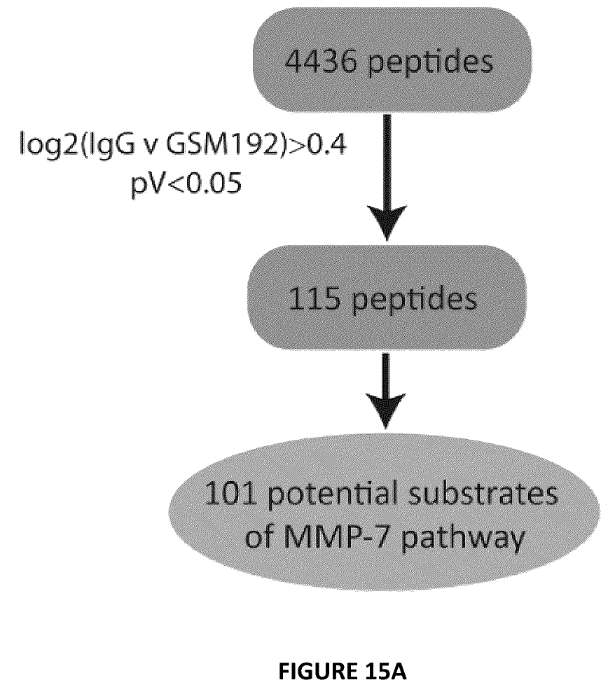

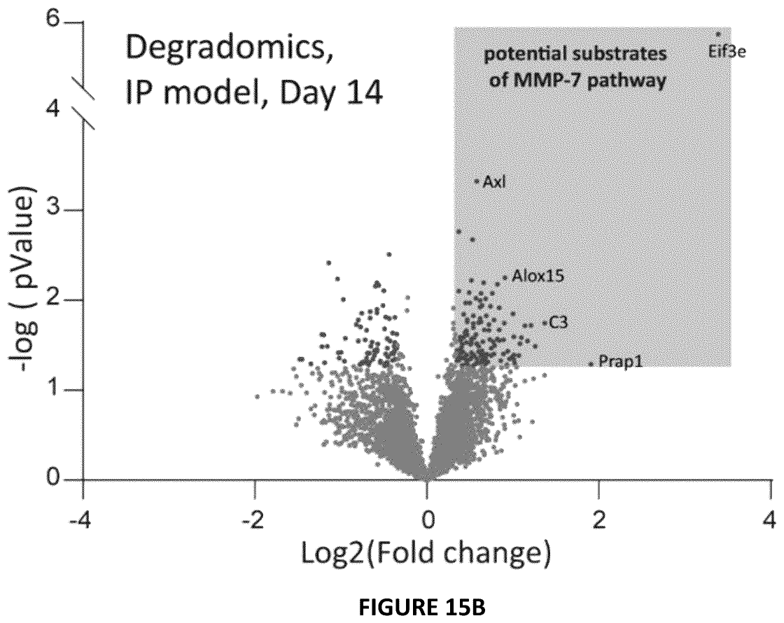

The inventors then aimed to explore the connection between active MMP-7 and the pathways depicted in FIG. 13 by examining MMP-7's direct substrates or those affected by its activity. To identify candidate MMP-7 pathway substrates in this context, the inventors applied the Terminal Amine Isotopic Labelling of Substrates (TAILS) method on lesions from the I.P. model at day14, assessing the change in cleavage event abundance after MMP-7 inhibition. Out of the 4,436 neo-N-terminal peptides detected, 115 peptides were identified as significant hits showing reduced abundance upon MMP-7 inhibition (log 2 (IgG v GSM-192)≥0.4; p≤0.05), corresponding to 101 proteins (FIG. 15A). Among these proteins, some demonstrated significant fold changes and low P values, including Eif3e, AXL, Prap1, Alox15, and C3 (FIG. 15B).

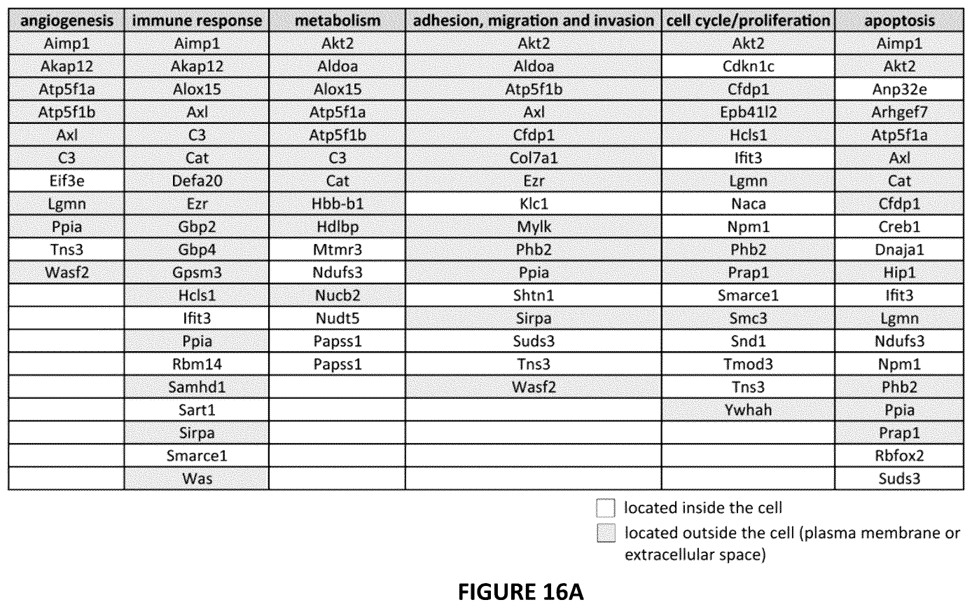

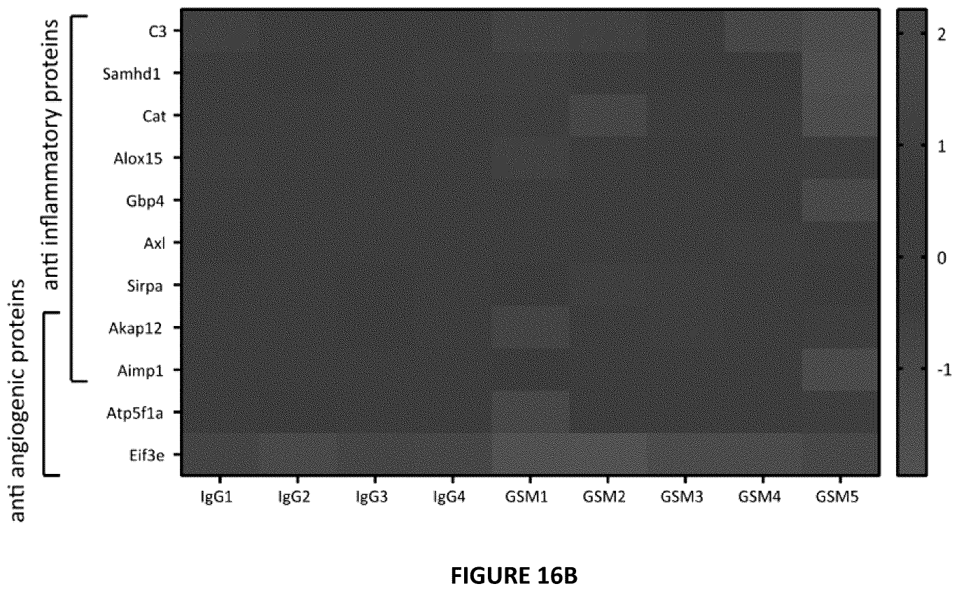

Utilizing the David analysis tool, the inventors categorized the significant hits that showed reduced abundance upon MMP-7 inhibition into six main process categories related to endometriosis based on their Gene Ontology (GO) pathways: angiogenesis, immune response, metabolism, adhesion/migration/invasion, cell cycle/proliferation, and cell death (FIG. 16A). The table also differentiates between proteins located inside the cell and those outside (plasma membrane or extracellular space). Based on this analysis and additional review of the literature, the inventors generated a heatmap for the proteins known to have anti-inflammatory or anti-angiogenesis functions (FIG. 16B).

These results link specific MMP-7 pathway substrates to the pathways identified in the RNA-Seq data, helping to elucidate MMP-7's role in the formation of endometriotic lesions.

### Example 11

### Eif3e is Upregulated Following MMP-7 Inhibition

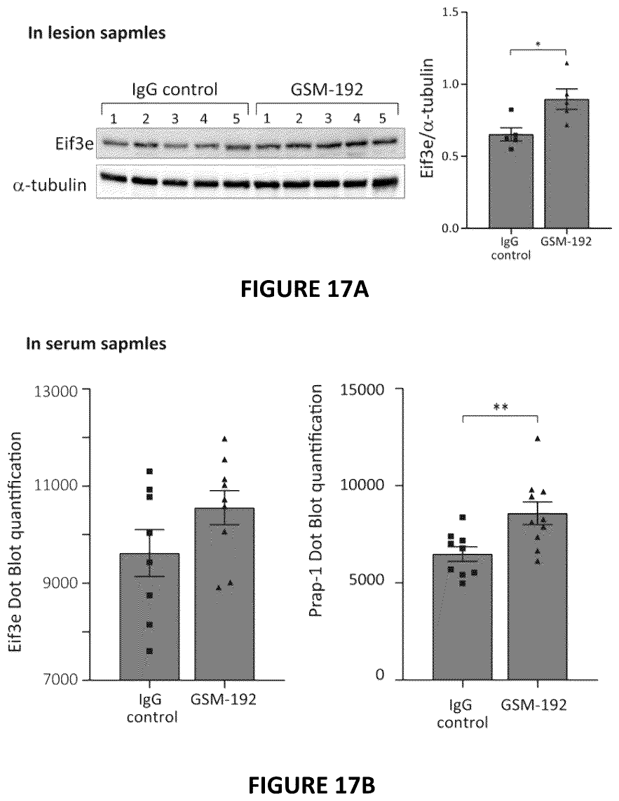

Eif3e was identified as a substrate in the MMP-7 pathway (FIGS. 15-16). Being an intracellular protein, it is probably not a direct substrate of MMP-7 but rather downstream of it. The fold change of this peptide in the degradomic analysis was notably high (log 2 (IgG vs GSM-192)=10.5), with a highly significant p-value (p<0.001). Consequently, the inventors investigated this protein via western blot to determine if MMP-7 influences its level in the lesion. Indeed, in FIG. 17A it is shown that lesions on day 21 from the I.P. model in mice treated with GSM-192 displayed higher levels of Eif3e compared to mice treated with the IgG control. This suggests that MMP-7 activity contributes to the breakdown of this protein. Eif3e acts as an anti-angiogenic and anti-migration factor, indicating that its degradation within the MMP-7 pathway could enhance angiogenesis and migration. In addition, dot blots of serum samples showed higher levels of Eif3e and Prap1 in the serum of GSM-treated mice, which verify the degradation of these substrates in the MMP-7 pathway.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1.-21. (canceled)

22. A method for treating endometriosis or ameliorating at least one symptom associated therewith in a subject in need thereof, the method comprising administering to said subject a pharmaceutical composition comprising a therapeutically effective amount of an agent capable of inhibiting an active site of a matrix metalloprotease (MMP-7) protein.

23. A method for treating a subject afflicted with endometriosis or being afflicted with at least one symptom associated therewith, the method comprising: (a) determining whether at least one endometrial lesion characterized by increased MMP-7 expression level compared to a healthy control subject is present in said subject; and (b) administering to the subject determined as having the at least one endometrial lesion of (a) a pharmaceutical composition comprising a therapeutically effective amount of an agent capable of inhibiting an active site of a matrix metalloprotease (MMP-7) protein.

24. The method of claim 23, wherein said determining is in a biopsy sample obtained or derived from said subject.

25. method of claim 23, wherein said MMP-7 expression level comprises mRNA expression level, protein level, or both.

26. The method of claim 22, wherein said inhibiting is by binding to at least one amino acid residue selected from the group consisting of: L181, A216, Y241, P246, Q247, N243, Y172, T180, P237, T240, H229, S101, N179, and any combination thereof, of said active site of the MMP-7 protein.

27. The method of claim 22, wherein said agent is an antibody or an antigen-binding portion thereof.

28. The method of claim 27, wherein said antibody or an antigen-binding portion thereof comprises three heavy chain complementarity determining regions (CDR-H) and three light chain CDRs (CDR-L), wherein: CDR-H1 comprises the amino acid sequence: GYTFTDYN (SEQ ID NO: 1), CDR-H2 comprises the amino acid sequence: HINPNNGGTF (SEQ ID NO: 2), CDR-H3 comprises the amino acid sequence: GGGLRRGP (SEQ ID NO: 3), CDR-L1 comprises the amino acid sequence: ASESFDSYGNTFVH (SEQ ID NO: 4), CDR-L2 comprises the amino acid sequence: LVSNLE (SEQ ID NO: 5), and CDR-L3 comprises the amino acid sequence: QQNNEDPYT (SEQ ID NO: 6).

29. The method of claim 6, wherein said antibody or antigen-binding portion thereof comprises a heavy chain comprising the amino acid sequence:
(SEQ ID NO: 7)
EVQLQQSGPELVKPGASVKIPCKASGYTFTDYNMDWMKQSHGKSLEWIGH
INPNNGGTFYNQKFKDKATFIVDKSSNTAYMELRSLTSEDTAVYFCARGG
GLRRGPFAYWGQGTLVTVS.

30. The method of claim 27, wherein said antibody or antigen-binding portion thereof comprises a light chain comprising the amino acid sequence:
(SEQ ID NO: 8)
DIVTQSPASLAVSLGQRATISCRASESFDSYGNTFVHWYQQKPGQPPKLL
IYLVSNLESGVPAGFRGRGSRTDFTLTIDPVEADDAATYYCQQNNEDPYT
FGGGTKLEIKRA.

31. The method of claim 27, wherein said inhibiting is with an inhibition constant (Ki) of between 10 and 200 nM.

32. The method of claim 26, wherein said binding is with a dissociation constant (Kd) of between 30 and 50 nM.

33. The method of claim 22, formulated for intravenous administration.

34. The method of claim 22, wherein said antibody is a humanized monoclonal antibody.

35. The method of claim 22, wherein said subject comprises at least one endometrial lesion characterized by increased amount of at least one cell type selected from the group consisting of: an epithelial cell, a T cell, a myofibroblast, a mature blood cell, an endothelial progenitor cell, and any combination thereof, compared to a healthy control subject.

36. The method of claim 35, wherein said: epithelial cell, T cell, mature blood cell, endothelial progenitor cell, myofibroblast, or any combination thereof, is characterized by increased expression of a gene encoding MMP-7, compared to a control cell.

37. The method of claim 35, wherein said mature blood cell is characterized by expression of CD31 (CD31+).

38. The method of claim 35, wherein said endothelial progenitor cell is characterized by expression of CD34 (CD34+).

39. The method of claim 22, wherein a treated subject is characterized by reduced: number of endometriotic lesions, rate of implantation of endometriotic lesions, weight of endometriotic lesions, or any combination thereof.

40. The method of claim 22, wherein a treated subject comprises at least one endometrial lesion characterized by increased expression of at least one gene associated with any one of: eosinophil migration, T cell migration, cellular defense response, myeloid dendritic cell activation, chemokine binding, lymphocyte migration, antigen processing and presentation of exogenous peptide, chemokine-mediated signaling pathway, immune response-regulating cell surface receptor signaling pathway, immunological synapse, myeloid leukocyte activation, any combination thereof, compared to a non-treated control subject, and optionally wherein said antigen processing and presentation of exogenous peptide comprises processing and presentation via MHC class II.

41. The method of claim 22, wherein a treated subject comprises at least one endometrial lesion characterized by decreased expression of at least one gene associated with any one of: epithelial cell differentiation, glycolysis and/or gluconeogenesis, glucose metabolic process, cellular carbohydrate biosynthetic process, cell-cell adherence junction, tight junction, glucan biosynthetic process, junctional membrane complex, and any combination thereof, compared to a non-treated control subject.
